(19) United States
(12) Patent Application Publication
(10) Pub. No.: US 2025/0295363 A1
(43) Pub. Date: Sep. 25, 2025

(54) BIOLOGICAL INFORMATION PROVIDING APPARATUS

(71) Applicant: Asahi Kasei Microdevices Corporation, Tokyo (JP)

(72) Inventor: Shigeru IWASA, Tokyo (JP)

(21) Appl. No.: 19/080,918

(22) Filed: Mar. 16, 2025

(30) Foreign Application Priority Data
Mar. 25, 2024 (JP) 2024-047885
Nov. 26, 2024 (JP) 2024-205786

(51) Int. Cl. A61B 5/00; A61B 5/053

(52) U.S. Cl. CPC A61B 5/721; A61B 5/053; A61B 5/6831; A61B 2560/0209

(57) ABSTRACT

Provided is a biological information providing apparatus including: a first biological information output unit and a second biological information output unit each including: a biological signal measurement unit which measures a biological signal through a pair of electrodes in contact with a living body, a bioimpedance signal measurement unit which measures a bioimpedance generated between the pair of electrodes to output a bioimpedance signal corresponding to the bioimpedance, a noise signal generation unit which generates, from the bioimpedance signal, a noise signal indicating a noise component included in the biological signal, and a biopotential signal output unit which removes a component of the noise signal from the biological signal to output a resultant signal as a biopotential signal; and a determination unit which outputs a determination signal based on a predetermined condition.

The contents of the following patent application(s) are incorporated herein by reference:
NO. 2024-047885 filed in JP on Mar. 25, 2024
NO. 2024-205786 filed in JP on Nov. 26, 2024.

### BACKGROUND

### 1. Technical Field

The present invention relates to a biological information providing apparatus.

### 2. Related Art

Patent Document 1 describes “an electrode system for reducing motion artifacts caused by the movement of electrodes disposed on a patient's skin when a medical worker diagnoses the patient by using electrocardiograms or the like”.

### PRIOR ART DOCUMENTS

### Patent Document

Patent Document 1: U.S. Patent Application Publication No.2003/0171661

### BRIEF DESCRIPTION OF THE DRAWINGS

FIG. 1 is an example of a schematic diagram illustrating a bioimpedance BioZ generated in a living body 500.

FIG. 2 is an example of a schematic view of a cross section in which a plurality of electrodes 150 are attached to a wrist of the living body 500.

FIG. 3 illustrates an example of a block diagram of a configuration of a biological information providing apparatus.

FIG. 4 is a flowchart illustrating an example of an operation of a determination unit 50.

FIG. 5 is a flowchart illustrating an example of an operation of a biological information output unit 100 that is a main biological information output unit.

FIG. 6 is a flowchart illustrating an example of an operation of a biological information output unit 200 that is a replica biological information output unit.

FIG. 7 is a flowchart illustrating an example of the operation of the determination unit 50 different from that in FIG. 4.

FIG. 8 is a flowchart illustrating an example of the operation of the determination unit 50 different from those in FIGS. 4 and 7.

FIG. 9 illustrates an example of a block diagram of a configuration of a biological information providing apparatus in which a configuration of a biological information output unit 300 that is a main biological information output unit is different from that of the biological information output unit 100.

FIG. 10 illustrates an example of a block diagram of a biological information providing apparatus including a biological information output unit 400.

FIG. 11A illustrates an example of grouping of electrodes 150A to 150N connected to the biological information output units 400.

FIG. 11B illustrates an example of grouping of the electrodes 150A to 150N connected to the biological information output units 400.

FIG. 11C illustrates an example of grouping of the electrodes 150A to 150N connected to the biological information output units 400.

FIG. 11D illustrates an example of grouping of the electrodes 150A to 150N connected to the biological information output units 400.

FIG. 12 is a flowchart illustrating an example of the operation of the determination unit 50 in an embodiment of FIG. 10.

FIG. 13 is a flowchart illustrating an example of the operation of the biological information output unit 400 in the embodiment of FIG. 10.

### DESCRIPTION OF EXEMPLARY EMBODIMENTS

The present invention will be described below through embodiments of the invention, but the following embodiments do not limit the invention according to the claims. In addition, not all of the combinations of features described in the embodiments are essential to the solution of the invention.

In the present specification, when referring to variations of the same element, for example, the element may be referenced with an alphabet attached, for example, an electrode 150A, an electrode 150B, or the like. On the other hand, these constituent elements may be collectively referred to as, for example, an electrode 150 or the like.

There is known a wristband-type controller, a myoelectric prosthesis, or the like that predicts a motion of a human by detecting a myoelectric potential generated immediately before the motion of the human and controls an object according to the predicted motion of the human. An apparatus such as a wristband-type controller that detects a motion of a living body such as a human is equipped with a biological sensor such as a myoelectric sensor, and senses biological information such as a myoelectric potential or a bioimpedance to predict biological information regarding the living body such as the motion of the living body.

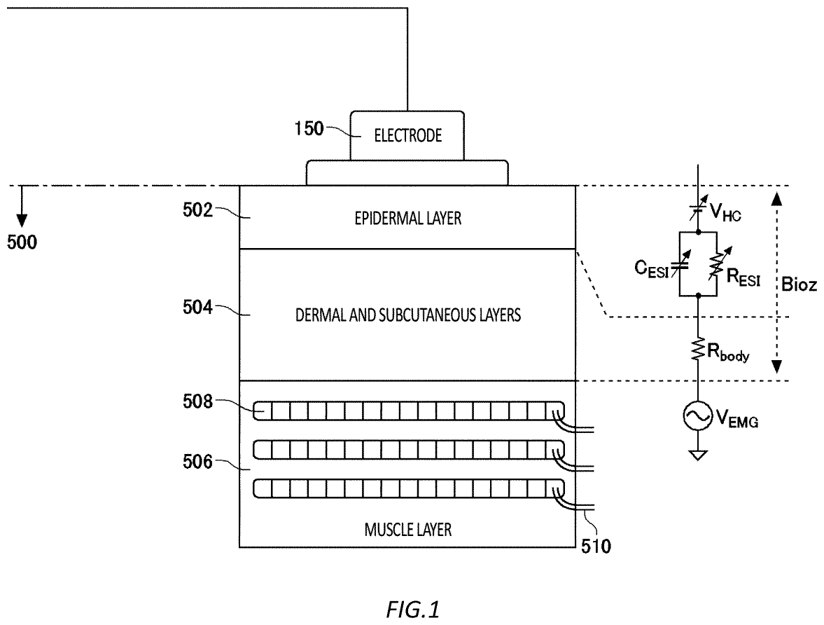

FIG. 1 is an example of a schematic diagram illustrating a bioimpedance BioZ generated in a living body 500. The living body 500 includes an epidermal layer 502, dermal and subcutaneous layers 504, and a muscle layer 506.

The living body 500 of the present embodiment is a human. However, the living body 500 may be another animal. In a case where the living body 500 is another animal, some skin structures may differ depending on the type of the living body 500.

In the measurement of the bioimpedance BioZ, a pair of electrodes 150 are brought into contact with and fixed to the living body 500, and a constant current is applied between the electrodes. Here, the pair of electrodes 150 may be fixed in contact with the same living body 500. In this case, the bioimpedance BioZ based on the body composition of the living body 500 can be read by reading a potential difference generated between the pair of electrodes 150.

In addition, for example, in a case where the living body 500 activates muscles, an action potential is generated by electrical excitation of cells in the muscle layer 506 of the living body 500. In the measurement of such an action potential, an impedance caused by the epidermal layer 502 and the dermal and subcutaneous layers 504 have an influence.

The epidermal layer 502 is an epidermis of the living body 500. For example, in a case where the living body 500 is a human, the epidermal layer 502 is a portion having an average thickness of about 0.2 mm in the skin of a portion other than a palm or the sole of a foot.

The epidermal layer 502 contributes as a half-cell potential V_{HC} and a variable resistance R_{ESI} and a variable capacitance C_{ESI} connected in series to the half-cell potential V_{HC} in the measurement of the bioimpedance BioZ by the electrode 150. The half-cell potential V_{HC} is an electrostatic potential generated at a portion where the electrode 150 and the epidermal layer 502 are in contact with each other. The component of the half-cell potential V_{HC} contributes, for example, as a component having a frequency of 20 Hz or less in the measurement of the bioimpedance BioZ. In the bioimpedance BioZ, the contribution of a contact impedance between the electrode 150 and the skin of the living body 500, that is, between the electrode 150 and the epidermal layer 502 is large. Furthermore, the epidermal layer 502 contributes as the variable resistance R_{ESI} and the variable capacitance C_{ESI} connected in series to the half-cell potential V_{HC}. The variable resistance R_{ESI} and the variable capacitance C_{ESI} vary significantly based on changes in the state of the skin surface and a contact state between the electrode 150 and the skin. This appears as a variation in the contact impedance between the electrode 150 and the skin of the living body 500 in the bioimpedance BioZ. In a state where the skin surface is dry, these impedances increase by about 10 times, and thus are expressed as variable resistances and capacitances in an equivalent circuit.

The dermal and subcutaneous layers 504 are layers such as a dermal layer, a subcutaneous tissue, and a fascia. In the measurement of the bioimpedance, the dermal and subcutaneous layers 504 contribute as a resistance R_{body}. Of the dermal and subcutaneous layers 504, the dermal layer is a site of the skin through which capillaries, lymphatic vessels, nerves, and the like pass, and is a portion formed inside the epidermal layer 502. For example, in a case where the living body 500 is a human, the dermal layer is a portion having an average thickness of about 2 mm. Of the dermal and subcutaneous layers 504, the subcutaneous tissue is formed further inside the dermal layer. The subcutaneous tissue is a portion that supports the epidermal layer 502 and the dermal layer, and is a portion having an average thickness of about 2 mm to about 9 mm. The subcutaneous layer mainly contains fat cells and includes large blood vessels and the like. Furthermore, the dermal and subcutaneous layers 504 include the fascia between the subcutaneous tissue and the muscle layer 506. The fascia has a thickness of about 1 mm and is a portion that generates an electrical resistance, although not as large as the skin. The resistance Rbody of the dermal and subcutaneous layers 504 is a resistance value obtained by adding the electrical resistances of the plurality of layers.

As a result, in the impedance measurement of the living body 500, what contributes as the bioimpedance BioZ is the variable resistance R_{ESI} and the variable capacitance C_{ESI} connected in parallel, and the resistance R_{body}.

The muscle layer 506 is a layer including a plurality of muscle fibers 508. The muscle fibers 508 are tissues that are activated by electrical signals transmitted through nerves 510. When the muscle fibers 508 move, action potentials are generated in the muscle fibers 508. In the muscle layer 506, a potential V_{EMG} (compound action potential V_{EMG}) obtained by adding (compounding) action potentials of a plurality of muscle fibers is generated according to the activities of a plurality of muscles. In the measurement of a myoelectric signal, such a compound action potential V_{EMG} is measured.

Since the action potential V_{EMG} is a potential generated by the activity of the living body, the action potential V_{EMG} is a potential difference that can be generated between a pair of electrodes 150 attached to the living body 500 even in a case where the energization from the outside is not performed. However, the myoelectric signal is a signal having a low potential, and thus in a case where the bioimpedance BioZ is measured together when such a myoelectric signal is measured, a signal which applies a potential difference between the pair of electrodes 150 is output from the outside of the living body 500.

The myoelectric signal is an example of a “biopotential signal”. The myoelectric signal may have a peak of amplitude in a frequency band higher than 20 Hz and up to 4 kHz, for example. As another example, the biopotential signal may be a signal indicating a potential generated by the activity of the living body such as cardiac activity, brain waves, or eye movements. Note that these biopotential signals are examples, and the biopotential signal is not limited to these signals as long as the biopotential signal is a signal based on the action potential of the living body 500 generated by the activity of the living body 500.

Here, in the measurement of the bioimpedance BioZ, in a case where the living body 500 moves, the electrode 150 is displaced with respect to the living body 500, or the living body 500 vibrates, so that a motion artifact (MA) generated at a contact point between the electrode 150 and the living body 500 may occur as measurement noise. In the measurement of the bioimpedance BioZ, it is known that a component proportional to the MA is included in a variation component of the bioimpedance BioZ.

The MA generated in a case where the living body 500 moves often has a peak at less than 20 Hz, for example, in a case where the living body 500 is a human. The influence of such an MA can be shielded by a high-pass filter. However, even when the frequency band in which the peak of the MA appears is a frequency band of 20 Hz or less, in a case where the peak value of the MA is large, the influence of the MA extending from the peak may appear in the frequency band of 20 Hz or more.

On the other hand, the vibration generated in the living body 500 is, for example, vibration or the like generated in a moving body, such as a train, a bus, or an airplane, on which a human is boarding. The MA generated when the living body 500 boards the moving body can be mixed directly as noise in a frequency band of 50 Hz or more and less than 200 Hz, for example. In this case, the MA is known to contribute as a variation of a capacitive component, and contributes as a component proportional to the variation component of the C_{ESI}. Furthermore, the variation of the bioimpedance BioZ due to the MA may have a frequency greater than a predetermined frequency (for example, 20 Hz).

Therefore, in the measurement of the variable capacitance C_{ESI}, by shielding a component in an appropriate frequency range and amplifying the shielded component, it is possible to read the variation of the capacitive element proportional to the MA, and eventually, it is possible to read the variation of the bioimpedance due to the MA. Hereinafter, a configuration of such a biological information providing apparatus capable of reading the variation of the variable capacitance C_{ESI} proportional to the MA will be described in detail.

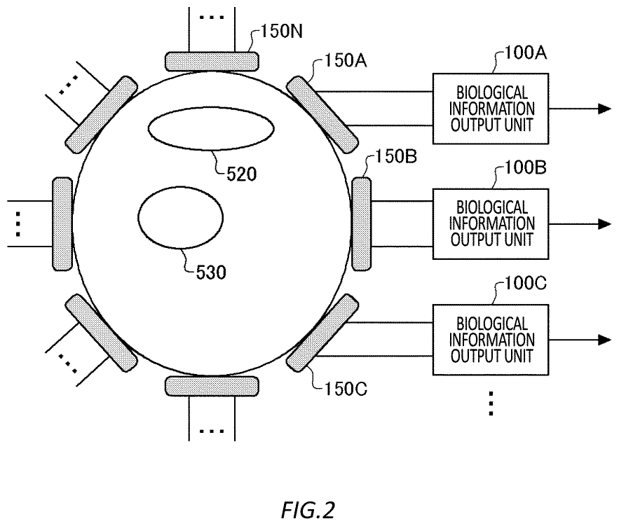

FIG. 2 is an example of a schematic view of a cross section in which a plurality of electrodes 150 are attached to the wrist of the living body 500. A biological information output unit 100 is connected to each of a plurality of pairs of electrodes 150. In the present embodiment, a case where the living body 500 is a human will be described as an example. A surface muscle 520 of a portion of the wrist closer to the wrist surface and a deep muscle 530 which is a muscle deeper away from the wrist surface are shown.

It is possible to recognize a gesture by wearing a bracelet type myoelectric sensor on a human wrist or forearm. In a case where the muscle of a hand is moved, a site where an action potential is generated is specified to specify which site of the muscle has moved and to specify the gesture of the hand. In order to specify the site where the action potential is generated, the plurality of electrodes 150 are disposed in pairs to surround the circumference of the wrist. Since the biological information output units 100 connected to the plurality of electrodes 150 are configured to form a sensor array, a site of activity can be specified from the action potential in the site corresponding to the activity of the living body 500 detected by the plurality of sensors. Specifically, distances from sensors at a plurality of locations to a muscle in which the action potential is generated, directions from the sensors to the muscle, and the like are specified to specify which muscle is moving. In the present embodiment, the plurality of electrodes 150 are disposed, for example, such that N pairs of electrodes 150 are separated at predetermined intervals in the circumferential direction of the wrist.

In the drawing, an example is illustrated in which the plurality of electrodes 150 are installed around the wrist or the forearm, but as another example, the plurality of electrodes 150 may be installed around the thigh. By installing the plurality of electrodes 150 around the thigh, deterioration or the like of muscles at a position where the myoelectric potential is generated can be detected, and a detection result can be used for applications such as sports engineering and rehabilitation.

The biological information output unit 100 is connected to each pair of the plurality of electrodes 150. For example, a biological information output unit 100A is connected to a pair of electrodes 150A, and a biological information output unit 100B is connected to a pair of electrodes 150B. In this manner, a biological information output unit 100N is connected to an Nth pair of electrodes 150N. The biological information output unit 100 detects biological information from each pair of the plurality of electrodes 150, and analyzes a distance from the electrode 150 to a site of activity, an angle, a correlation between the electrode 150 and the site of activity, and/or the like from the detected biological information, thereby specifying the site where the action potential is generated.

In a case where the wrist of the living body 500 is moved, the surface muscle 520 (for example, flexor pollicis longus) expands and contracts, and an action potential is generated in the surface muscle 520. In a case where the finger of the living body 500 is moved, a site more distal to the measurement site is moved, so that the deep muscle 530 (for example, flexor digitorum superficialis) at a deeper site expands and contracts, and an action potential is generated in the deep muscle 530. In this manner, the bracelet type myoelectric sensor is attached to the wrist or forearm, and the generation positions of the action potential at different sites in the wrist are specified, whereby the gesture of the finger can be specified.

As an example of measurement of other than the myoelectric potential, it is effective to

measure by installing a plurality of electrodes 150 for use in brain wave measurement, for example. Since a brain function often varies depending on a position in the brain, specifying the position of the source of brain waves in the brain can be helpful in analyzing the brain waves.

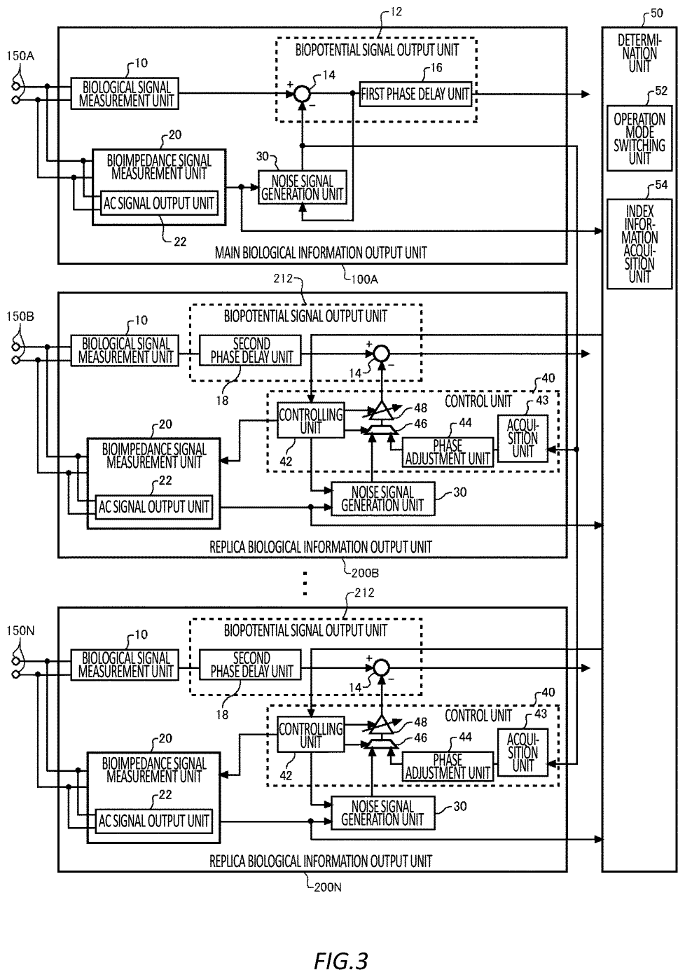

FIG. 3 illustrates an example of a block diagram of a configuration of the biological information providing apparatus. The biological information providing apparatus includes the biological information output unit 100, one or more biological information output units 200, and a determination unit 50. In the present embodiment, the biological information output unit 100A is connected to the pair of electrodes 150A, and a biological information output unit 200B is connected to the pair of electrodes 150B. In the drawing, the N pairs of electrodes 150 are illustrated similarly to that in the embodiment of FIG. 2. A biological information output unit 200N is connected to the electrodes 150N.

In the present embodiment, since the biological information output unit 100 and the biological information output unit 200 have different internal configurations, different reference numerals are attached to the respective biological information output units. The biological information output unit 100 includes a biological signal measurement unit 10, a bioimpedance signal measurement unit 20, a noise signal generation unit 30, and a biopotential signal output unit 12. The biological information output unit 200 includes the biological signal measurement unit 10, the bioimpedance signal measurement unit 20, the noise signal generation unit 30, a control unit 40, and a biopotential signal output unit 212. In addition, due to the difference in function between the biological information output unit 100 and the biological information output unit 200, the biological information output unit 100 may be referred to as a “main biological information output unit” or a “first biological information output unit”. On the other hand, the biological information output unit 200 may be referred to as a “replica biological information output unit 200” or a “second biological information output unit”.

The biological signal measurement unit 10 measures a biological signal through a pair of electrodes 150 in contact with the living body 500. The biological signal measurement unit 10 may amplify the compound action potential V_{EMG} to output an analog-digital converted signal as the biological signal.

The bioimpedance signal measurement unit 20 measures the bioimpedance generated between the pair of electrodes 150 to output a bioimpedance signal corresponding to the bioimpedance BioZ. The bioimpedance signal measurement unit 20 outputs the bioimpedance signal to the noise signal generation unit 30 and the determination unit 50. The bioimpedance signal measurement unit 20 includes an AC signal output unit 22.

The AC signal output unit 22 applies an AC signal to the pair of electrodes 150 in order for the bioimpedance signal measurement unit 20 to measure the bioimpedance BioZ. For example, the AC signal output unit 22 includes a DC constant voltage source or a constant current source, and a mixer for generating an AC constant voltage signal. As another example, an AC power source with a square wave or sine wave and a resistor for limiting the amplitude of a signal are included.

The AC signal output unit 22 may apply an AC signal as a differential signal to the pair of electrodes 150. In this case, the bioimpedance signal measurement unit 20 measures the bioimpedance BioZ by detecting an impedance with respect to the differential signal. In another example, the AC signal output unit 22 applies, to one of the pair of electrodes 150, a waveform signal with a sine wave or square wave-shaped potential difference from a predetermined reference potential. In this case, the bioimpedance signal measurement unit 20 may include an amplifier and a single-ended to differential conversion circuit, and measure the bioimpedance by extracting a differential signal from a single-ended signal.

The noise signal generation unit 30 generates, from the bioimpedance signal, a noise signal indicating a noise component included in the biological signal. The noise signal generation unit 30 generates a signal proportional to MA by multiplying the bioimpedance signal by a coefficient. Here, the noise signal generation unit 30 may be an adaptive filter that adapts coefficients by feedback control according to the output from the biological information output unit 100A to output a filtered signal. Therefore, the noise signal generation unit 30 may include an adaptive filter that generates a noise signal based on the bioimpedance signal and the coefficient. The motion artifact MA is a noise component included in a compound component of action potentials from each of a plurality of muscle fibers constituting the living body 500 in the measurement of the myoelectric potential. The adaptive filter reduces variation of the output from the biological signal measurement unit 10 due to the type of MA or the like, and converges the output of the biopotential signal output unit 12 to a signal reflecting desired biological information.

In the biological information providing apparatus configured as described above, when the number of the biological information output units 100 and the number of the biological information output units 200 are large, there is a high possibility that which site of the muscle has moved can be specified more accurately from the biopotential signal output from each unit. On the other hand, power consumption increases as the biological information output unit 100 and the biological information output unit 200 operate. Therefore, it is desired to suppress the power consumption in the biological information providing apparatus including the biological information output unit 100 and the biological information output unit 200.

Here, the noise signal includes a noise component generated by the vibration generated in the living body 500. Such noise components are components included in respective biological signals of the biological information output unit 100 and the biological information output unit 200, and may be similar. In addition, the accuracy required to specify which site of the muscle has moved varies depending on applications and the like. That is, it may not always be necessary for the accuracy to be high. In this regard, in the biological information providing apparatus according to the present embodiment, the noise signal used by the biological information output unit 100 and the biological information output unit 200 is shared according to a predetermined condition in consideration of required accuracy and the like, and power consumption for generating the noise signal is suppressed. Here, in terms of sharing the signal regarding noise, the signal shared by the biological information output unit 100 and the biological information output unit 200 may be the output of the bioimpedance signal measurement unit 20 having a correlation with the noise signal. However, as compared with a case where the output of the bioimpedance signal measurement unit 20 is shared, in a case where the noise signal is shared, it is not necessary to operate the noise signal generation unit 30 in each biological information output unit 200, and power consumption may be suppressed more greatly.

In order to implement this, the noise signal generation unit 30 of the biological information output unit 100 outputs a noise signal to the signal line connected to the biological information output unit 200. A bus, a hub, or the like may be connected to the signal line, and the biological information output unit 200 may selectively acquire the noise signal from the biological information output unit 100.

The determination unit 50 outputs a determination signal for determining whether to share the noise signal used in the biological information output unit 100 and the biological information output unit 200, based on a predetermined condition. The determination unit 50 may output a determination signal indicating a first determination result or a second determination result according to a predetermined condition. The determination unit 50 includes an operation mode switching unit 52 and an index information acquisition unit 54.

The determination unit 50 may output the determination signal indicating the first determination result or the second determination result, based on a condition of the similarity degree of the bioimpedance signals as the predetermined condition. Specifically, for example, the determination unit 50 receives the bioimpedance signal from the bioimpedance signal measurement unit 20 of each of the biological information output unit 100A and the biological information output units 200B to 200N. The determination unit 50 compares the bioimpedance signal of the biological information output unit 100A with the bioimpedance signal of each of the biological information output units 200B to 200N, and determines whether or not the bioimpedance signals have a similarity degree equal to or greater than a predetermined similarity degree. Since the MA depends on a contact state between the electrode 150 and the living body 500, the similarity degree of the bioimpedance signals being equal to or greater than the predetermined similarity degree indicates that the similarity in the contact state between the electrode 150 and the living body 500 is equal to or greater than a certain level.

The similarity degree determination performed by the determination unit 50 may be performed by comparing spectral components of the bioimpedance signals. The determination unit 50 uses a method such as a fast Fourier transform (FFT) to convert a signal component over time into a frequency component for comparison. The determination unit 50 may compare the shape or intensity of the spectrum at each frequency component to determine the similarity degree. Alternatively, the similarity degree determination may be based on similarity degree comparison performed by comparing the positions of the peaks or inflection points of signal components or the like using a method such as a cross-correlation function for time-domain signals. For example, in a case where the absolute value of a difference between these positions is within a predetermined value, the similarity degree can be determined to be equal to or greater than the predetermined similarity degree. Alternatively, the slope of the signal, the degree of variation, and the overall shape of the signal component in the bioimpedance signal may be compared using a method such as a correlation function. For example, in a case where a difference in the slope of the signal is within a predetermined value, the similarity degree can be determined to be equal to or greater than the predetermined similarity degree.

In a case where the similarity degree between the bioimpedance signal of the biological information output unit 100 and the bioimpedance signal of the biological information output unit 200 is equal to or greater than the predetermined similarity degree, the determination unit 50 may output a determination signal indicating the first determination result to the control unit 40 of the biological information output unit 200 according to a predetermined condition. On the other hand, in a case where the similarity degree between the bioimpedance signal of the biological information output unit 100 and the bioimpedance signal of the biological information output unit 200 is less than the predetermined similarity degree, the determination unit 50 may output a determination signal indicating the second determination result according to the predetermined condition.

In the present embodiment, the bioimpedance signal is input from the bioimpedance signal measurement units 20 of the biological information output unit 100 and the biological information output unit 200 to the determination unit 50. The determination unit 50 determines the similarity in the contact state between the electrode 150 and the living body 500, based on the similarity degree of the input bioimpedance signals.

In another example, each of the biological information output unit 100 and the biological information output unit 200 inputs, to the determination unit 50, the noise signal generated from the noise signal generation unit 30. In this case, the determination unit 50 may compare the similarity degree of the noise signals to determine the similarity in the contact state between the electrode 150 and the living body 500.

As still another example, the biopotential signal reflecting the biological information output from the biological information output unit 100 and the biological information output unit 200 may be input to the determination unit 50. The biological information output unit 100 and the biological information output unit 200 connected to the electrodes 150 disposed on the site of the living body such as the wrist in the circumferential direction constitute a sensor array.

Although the biopotential signals output from the biological information output unit 100 and the biological information output unit 200 have angle dependency, the biopotential signals are signals from the electrodes 150 disposed at equal intervals, and noise removal is performed on each of the biopotential signals, so that in a case where there is similarity of the noise signals, correlation also appears in the biopotential signals. Alternatively, in a case where the influence of the MA remains in the biopotential signal, the similarity degree in the influence of the MA can be confirmed by comparing the similarity degree of the biopotential signals. Accordingly, the biological information output unit 200 may determine whether or not to use the noise signal of the biological information output unit 100. Therefore, the determination unit 50 can also determine whether or not the biological information output unit 200 is to use the noise signal of the biological information output unit 100, by comparing the similarity degree of the biopotential signals.

That is, in a case where the similarity degree between the biopotential signal of the biological information output unit 100 and the biopotential signal of the biological information output unit 200 is equal to or greater than the predetermined similarity degree, the determination unit 50 may output the determination signal indicating the first determination result according to the predetermined condition. In a case where the similarity degree between the biopotential signal of the biological information output unit 100 and the biopotential signal of the biological information output unit 200 is less than the predetermined similarity degree, the determination unit 50 may output the determination signal indicating the second determination result according to the predetermined condition. The determination unit 50 may output the determination signal indicating the first determination result and the second determination result to the control unit 40 of the biological information output unit 200.

Therefore, in order to perform this determination, the bioimpedance signals of the biological information output unit 100 and the biological information output unit 200 or the biopotential signals of the biological information output unit 100 and the biological information output unit 200 may be input to the determination unit 50. The determination unit 50 may output the determination signal based on the input bioimpedance signal or biopotential signal.

The determination unit 50 may output the determination signal indicating the first determination result or the second determination result, based on a condition of the magnitude of vibration as the predetermined condition. Specifically, the index information acquisition unit 54 acquires, as index information, at least one of position information of the living body 500, vibration information including the magnitude of vibration of the living body 500, or acceleration information of the living body 500.

In this case, the index information acquisition unit 54 includes at least one or more of combinations of a GPS, an inertial measurement unit (IMU), an acceleration sensor, a magnetic sensor, a gyroscope, and the like for acquiring the position information, movement information, the vibration information, and/or the acceleration information. For example, in a case where the index information acquisition unit 54 includes a GPS, it is possible to read geographical position information and movement information such as vehicle boarding, walking, or stationary state. For example, in a case where the index information acquisition unit 54 includes a gyroscope, it is possible to detect an axis on which the biological information providing apparatus is rotating, and to estimate a movement state. For example, in a case where the index information acquisition unit 54 includes a magnetic sensor, the movement direction of the biological information providing apparatus can be estimated by detecting geomagnetism. Note that the vibration information of the living body includes, for example, information regarding the magnitude of vibration of the living body and/or frequency information of the vibration.

As the predetermined condition, the determination unit 50 may output a determination signal indicating the first determination result in a case where the index information indicates that the magnitude of the vibration of the living body is equal to or greater than a predetermined magnitude, and may output a determination signal indicating the second determination result according to the predetermined condition in a case where the index information indicates that the magnitude of vibration of the living body is less than the predetermined magnitude. For example, in a case where the magnitude of vibration of the living body 500 is greater than the predetermined magnitude, the living body 500 may be in a state of moving by train. In the case of moving by train, the MA is patterned, and similar noise signals are removed by the main biological information output unit and the replica biological information output unit, whereby the influence of the MA from the biopotential signal is easily removed. Therefore, in the present embodiment, in a case where the vibration is large, the determination unit 50 outputs the determination signal indicating the first determination result and causes the replica biological information output unit to use the noise signal output by the noise signal generation unit 30 of the main biological information output unit.

However, the determination by the magnitude of vibration in the present embodiment is merely an example, and the determination unit 50 may determine the first determination result and the second determination result by various pattern analyses on the bioimpedance signal. For example, the determination unit 50 may measure the bioimpedance signal for a predetermined period, and in a case where repetitive vibration appears at a predetermined amplitude and cycle for a predetermined number of times or more, detect a vehicle boarding state and output the determination signal indicating the first determination result. In this case, the determination unit 50 may output the determination signal indicating the second determination result in a case where the repetitive vibration at the predetermined amplitude and cycle for the predetermined number of times does not appear in the bioimpedance signal.

The determination unit 50 may output the determination signal indicating the first determination result or the second determination result, based on, as the predetermined condition, a condition corresponding to the operation mode of the biological information providing apparatus based on the reliability required for the biological information. Specifically, as the predetermined condition, in the case of a first operation mode in which the biopotential signal is output with the reliability required for the biological information being first reliability, the determination unit 50 may output the determination signal indicating the first determination result, and in the case of a second operation mode in which the biopotential signal is output with the reliability required for the biological information being second reliability higher than the first reliability, the determination unit 50 may output the determination signal indicating the second determination result. The operation mode switching unit 52 may switch these operation modes.

In a case where higher reliability is required, the number of measurement points for measuring the noise signal is increased to allow the use of the noise signal based on the bioimpedance signal measured at each position of the electrodes 150B to 150N. In other words, the biological information output units 200 that are a larger number of replica biological information output units are caused to remove the influence of the MA by using the noise signals output by their own noise signal generation units 30, whereby the reliability can be increased. This is a trade-off with reduction in power consumption by causing a larger number of replica biological information output units to use the noise signal output by the noise signal generation unit 30 of the main biological information output unit. Therefore, the determination unit 50 can output the determination signal in which the combination of the number and positions of the replica biological information output units that use the noise signals output by their own noise signal generation units 30 is adjusted according to the desired reliability and reduction in power consumption.

The biopotential signal output unit 12 removes the component of the noise signal from the biological signal to output the resultant signal as the biopotential signal. Specifically, the biopotential signal output unit 12 includes a subtractor 14, and the subtractor 14 subtracts the noise signal from the biological signal. Furthermore, the biopotential signal output unit 12 includes a first phase delay unit 16 (described later), which is another channel, in order to align the phase with the biopotential signal output from the biological information output unit 200. The first phase delay unit 16 adjusts the phase of the signal from which the influence of noise has been removed by the subtractor 14, to output the phase-adjusted signal as the biopotential signal of the biological information output unit 100A. In this manner, the biopotential signal output unit 12 may add or subtract the biological signal and the noise signal to output the resultant signal as the biopotential signal.

The subtractor 14 outputs a signal obtained by subtracting the noise signal from the input biological signal. Specifically, the subtractor 14 subtracts a component proportional to the MA from the biological signal input by the biological signal measurement unit 10, thereby outputting a signal from which the influence of the MA has been removed.

The first phase delay unit 16 synchronizes the biopotential signal output from the biopotential signal output unit 212 of the biological information output unit 200 with the biopotential signal output from the biopotential signal output unit 12 of the biological information output unit 100A. As described later, a second phase delay unit 18 receives the noise signal from the biological information output unit 100A, and performs phase adjustment with the biological signal of the biological information output unit 200 by delaying the phase. Accordingly, the biopotential signal output from the biological information output unit 200 becomes a delayed signal, and thus the first phase delay unit 16 performs phase adjustment by delaying the biopotential signal output from the biological information output unit 100A with respect to the biopotential signal output from the biological information output unit 200.

For example, in a case where the biological information output unit 100A is implemented by an integrated circuit (IC) operating under a reference clock, the first phase delay unit 16 can be implemented using a flip-flop. This is because the flip-flop has a property as an order circuit that holds information of a timing indicated by a specific clock and outputs a signal based on the information from a timing indicated by a next clock. Alternatively, the first phase delay unit 16 can be implemented by a first-in-first-out (FIFO) memory having a characteristic of storing data in a certain order and outputting first input data first. Alternatively, the first phase delay unit 16 may be implemented using a timer. In this manner, the first phase delay unit 16 can be implemented by using an appropriate delay circuit. By using the first phase delay unit 16, a phase difference in the output signal between the different ICs is compensated, and accuracy in performing gesture recognition using the myoelectric signal is improved. Note that the first phase delay unit 16 is a constituent element that can be omitted in a case where the phase difference between the biopotential signals output from the biological information output unit 100 and the biological information output unit 200 is not large.

The biological information output unit 200 is common with the biological information output unit 100 in that the biological information output unit 200 includes the biological signal measurement unit 10, the bioimpedance signal measurement unit 20, and the noise signal generation unit 30. Therefore, hereinafter, the control unit 40 and the biopotential signal output unit 212 included in the biological information output unit 200B will be described.

The control unit 40 controls the noise signal output to the biopotential signal output unit 212, based on the determination signal input from the determination unit 50. Specifically, in a case where the determination signal indicates the first determination result, the control unit 40 inputs the noise signal generated by the noise signal generation unit 30 of the biological information output unit 100A to the biopotential signal output unit 212 of the biological information output unit 200. In a case where the determination signal indicates the second determination result, the control unit 40 performs control such that the noise signal generated by the noise signal generation unit 30 of the biological information output unit 200 is input to the biopotential signal output unit 212 of the biological information output unit 200. As described above, in a case where the determination unit 50 determines that the predetermined condition is satisfied and outputs the determination signal indicating the first determination result, the biological information output unit 200 serving as the replica biological information output unit removes the influence of the MA from the biopotential signal by using the noise signal (which is an example of the “external noise signal” input from the outside of the replica biological information output unit) of the biological information output unit 100A serving as the main biological information output unit. The replica biological information output unit of the present embodiment is referred to as a “replica” in that noise removal may be performed by using the noise signal generated by the main biological information output unit instead of the noise signal generated by the corresponding replica biological information output unit.

Furthermore, in a case where the determination signal indicates the first determination result, the control unit 40 controls at least one of the bioimpedance signal measurement unit 20 or the noise signal generation unit 30 of the biological information output unit 200 so as to suppress power consumption in at least one of the bioimpedance signal measurement unit 20 or the noise signal generation unit 30 of the biological information output unit 200. For example, the control unit 40 performs control to suppress power consumption by executing at least one of stop of supply of the biological signal by the bioimpedance signal measurement unit 20 or stop of generation of the noise signal by the noise signal generation unit 30. As another example in which the control unit 40 suppresses power consumption in a case where the determination signal indicates the first determination result, in a case where the bioimpedance signal measurement unit 20 includes the AC signal output unit 22, the control unit 40 may suppress the power consumption by stopping the supply of the AC signal by the AC signal output unit 22.

As still another example, the control unit 40 may intermittently operate the bioimpedance signal measurement unit 20 and the noise signal generation unit 30. Here, “intermittently operate” means causing a target apparatus, circuit, or the like to perform an operation of switching between a normal operation and a standby operation at predetermined time intervals. Accordingly, the control unit 40 can reduce power consumption as compared with a case where the bioimpedance signal measurement unit 20 and the noise signal generation unit 30 are continuously operated.

As still another example, the control unit 40 may slow down the operations of the bioimpedance signal measurement unit 20 and the noise signal generation unit 30. This means that the control unit 40 delays an operation switching timing and/or a time required for switching in circuits involving switching operations, such as switching circuits or logic circuits, included in the bioimpedance signal measurement unit 20 and the noise signal generation unit 30. Accordingly, an operating current accompanying signal switching is suppressed, and power consumption can be reduced.

This is because the biological information output unit 200 serving as the replica biological information output unit uses the noise signal of the biological information output unit 100A serving as the main biological information output unit, so that it is not necessary to generate the noise signal in the biological information output unit 200. In this case, since the operations of the bioimpedance signal measurement unit 20 and the noise signal generation unit 30 on the biological information output unit 200 side can be omitted, the power consumption can be reduced by stopping at least one of these elements. The control unit 40 includes a controlling unit 42, an acquisition unit 43, a phase adjustment unit 44, a selector 46, and an amplification unit 48.

In a case where the determination signal from the determination unit 50 is input to the control unit 40, the controlling unit 42 controls the bioimpedance signal measurement unit 20, the noise signal generation unit 30, the selector 46, and the amplification unit 48. Specifically, a signal for performing the following control is output.

In a case where the determination signal indicates the first determination result, the controlling unit 42 outputs a signal for stopping the operation of at least one of the bioimpedance signal measurement unit 20 or the noise signal generation unit 30. In this case, the controlling unit 42 may also output a signal for stopping the operation of the AC signal output unit 22. The controlling unit 42 outputs, to the selector 46, a signal for the selector 46 to select the noise signal of the biological information output unit 200 that is the replica biological information output unit. In addition, in a case where the noise signal of the replica biological information output unit is output to the biopotential signal output unit 212, the controlling unit 42 outputs a signal for controlling the amplification unit 48 so as to amplify the noise signal at an appropriate amplification factor.

On the other hand, in a case where the determination signal indicates the second determination result, the controlling unit 42 does not output the signal for stopping the operations of the bioimpedance signal measurement unit 20, the AC signal output unit 22, and the noise signal generation unit 30. In this case, the controlling unit 42 also does not output the signal for stopping the operation of the AC signal output unit 22. The controlling unit 42 outputs, to the selector 46, a signal for the selector 46 to select the noise signal of the biological information output unit 100A that is the main biological information output unit. In addition, in a case where the noise signal of the main biological information output unit is output to the biopotential signal output unit 212, the controlling unit 42 outputs a signal for controlling the amplification unit 48 so as to amplify the noise signal at an appropriate amplification factor.

The acquisition unit 43 acquires, from the signal line, the noise signal output by the noise signal generation unit 30 of the biological information output unit 100. The acquisition unit 43 may periodically acquire the noise signal from the biological information output unit 100. The noise signal output from the biological information output unit 100 may be attached with information, such as header information, that allows for identification of the noise signal as being output from the biological information output unit 100. The acquisition unit 43 may have a configuration to determine, based on a header or the like, that the noise signal is a signal from the biological information output unit 100.

Here, in a case where the biological information output unit 100A and the biological information output unit 200 are provided in different ICs, the biological information output unit 100A and the biological information output unit 200 can be synchronized with different reference clocks, respectively. In a case where data is passed from the biological information output unit 100A to the biological information output unit 200, the phase adjustment unit 44 performs timing compensation by transferring the data synchronized with the biological information output unit 100A to a clock system on the biological information output unit 200 side. At this time, a signal delay of about 1/fs may occur with respect to a sampling frequency fs at the time of data fetching. For example, the phase adjustment unit 44 is implemented by using a delay circuit such as a flip-flop.

The selector 46 selects a noise signal to be input to the amplification unit 48, based on the signal from the controlling unit 42. The selector 46 may switch the noise signal input to the amplification unit 48 according to the level of the signal from the controlling unit 42. The selector 46 switches the noise signal input to the amplification unit 48 between the noise signal input from the noise signal generation unit 30 of the biological information output unit 200 and the noise signal input from the biological information output unit 100A via the acquisition unit 43 and the phase adjustment unit 44, according to the level of the signal from the controlling unit 42.

The amplification unit 48 switches the amplification factor of the noise signal to be output to the biopotential signal output unit 212, according to the signal from the controlling unit 42. For example, a contact state between the living body 500 and the electrode 150 to which the biological information output unit 100 that is the main biological signal output unit is connected may be different from a contact state between the living body 500 and the electrode 150 to which the biological information output unit 200 that is the replica biological signal output unit is connected. This appears as a difference in the level of the bioimpedance signal input to the determination unit 50. Therefore, the controlling unit 42 controls the amplification unit 48 to amplify the noise signal with the amplification factor of the noise signal corresponding to the level ratio of the bioimpedance signal, based on the signal from the determination unit 50.

As described above, the biological information output unit 200 includes the amplification unit 48 which amplifies or attenuates the noise signal, which serves as an input to the biopotential signal output unit 212 of the biological information output unit 200, with the amplification factor based on the level ratio between the bioimpedance signal measured by the bioimpedance signal measurement unit 20 of the biological information output unit 100 and the bioimpedance signal measured by the bioimpedance signal measurement unit of the biological information output unit 200, and then outputs the resultant signal to the biopotential signal output unit 212 of the biological information output unit 200. Accordingly, even in a case where the influence of the MA is removed from the biological signal based on the noise signal of a different IC, the biopotential signal output unit 212 can amplify the influence of the signal component proportional to the MA to an appropriate amplitude and remove the influence. Note that the amplification unit 48 is a configuration that can be omitted in a case where the level difference in the biopotential signal and the noise signal between the biological information output unit 100 and the biological information output unit 200 is not large.

The biopotential signal output unit 212 includes the second phase delay unit 18 and the subtractor 14. The second phase delay unit 18 synchronizes the biological signal from the biological signal measurement unit 10 of the biological information output unit 200 with the noise signal generated by the noise signal generation unit 30 of the biological information output unit 100A. By providing the second phase delay unit 18, the biological signal and the noise signal proportional to the MA are synchronized with each other, and the effect of suppressing the influence of the noise proportional to the MA on the biopotential signal can be improved. Similarly to the first phase delay unit 16, the second phase delay unit 18 may be implemented by using a flip-flop, a FIFO, a timer, and/or the like. Note that the second phase delay unit 18 is a constituent element that can be omitted in a case where the phase difference between the biological signal and the noise signal proportional to the MA is not large.

The configurations of the biological information output unit 100A and the biological information output units 200B to 200N have been described above. In the present embodiment, the biological information output unit 100A that is one main biological information output unit, and N-1 biological information output units 200B to 200N are provided for the N pairs of electrodes 150, respectively. However, it is sufficient that the number of the main biological information output units 100 is one or more, and the number is not limited to one. In a case where the number of pairs of electrodes is N, the number of biological information output units 100 that are the main biological information output units may be any one of 1 to N. It is sufficient that after selection of the number of main biological information output units, the biological information output units 200 that are the replica biological information output units are provided as many as the number satisfying (number of replica biological information output units)=N−(number of main biological information output units). In this case, it is sufficient that the control unit 40 of the replica biological information output unit is connected to the noise signal generation unit 30 of any one of the main biological information output units having similar noise signals.

The biological information providing apparatus may include a main biological information output unit and a plurality of replica biological information output units, and may include a band for holding a pair of electrodes 150 for each biological information output unit and being worn on the living body 500. The band may hold the pair of electrodes 150 at equal intervals along the circumferential direction of the wrist of the living body 500. The band can be used to maintain the position between the electrodes 150 and the contact state between the living body 500 and the electrode 150.

In addition, the biological information providing apparatus may include a plurality of main biological information output units. In this case, the biological information providing apparatus may include a plurality of main biological information output units and a plurality of replica biological information output units, and the replica biological information output unit may be assigned to each of the plurality of main biological information output units, based on a similarity degree of bioimpedance signals derived in advance through an experiment, a calibration process, or the like. Since the similarity degree of the noise signals tends to increase as a distance between the electrodes 150 decreases, a predetermined number of biological information output units among the biological information output units arranged at equal intervals may be set as the main biological information output units.

In this case, at least one of the plurality of replica biological information output units may be associated with each of the plurality of main biological information output units. That is, the control units 40 of the plurality of biological information output units 200 are connected to the respective bioimpedance signal measurement units 20 of the plurality of biological information output units 100. This connection may be performed via the above-described signal line having a bus or the like. In a case where the determination signal indicates the first determination result, the control unit 40 of each of the plurality of biological information output units 200 may perform control such that the noise signal generated by the noise signal generation unit 30 of the biological information output unit 100 associated with the corresponding biological information output unit 200 is input to its own biopotential signal output unit 212. In this case, the biological information providing apparatus may include a band for holding a pair of electrodes 150 of each of the plurality of main biological information output units and the plurality of replica biological information output units and being worn on the living body 500. The band may hold the pair of electrodes 150 at equal intervals along the circumferential direction of the wrist of the living body 500. The band can be used to maintain the position between the electrodes 150 and the contact state between the living body 500 and the electrode 150.

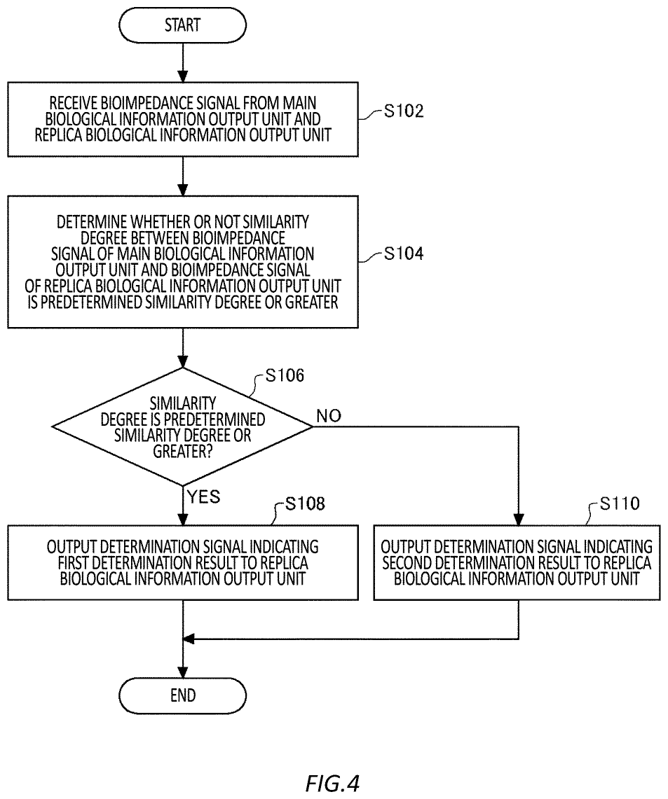

Next, with reference to FIGS. 4 to 7, the operations of the determination unit 50, the main biological information output unit, and the replica biological information output unit will be described for some embodiments. FIG. 4 is a flowchart illustrating an example of the operation of the determination unit 50. The operation of the determination unit 50 of the present embodiment includes steps S102 to S110.

The determination unit 50 receives the bioimpedance signal from the biological information output unit 100 and the biological information output unit 200 (S102). The determination unit 50 determines whether or not a similarity degree between the bioimpedance signal of the biological information output unit 100A, which is the main biological information output unit, and the biological information output unit 200, which is the replica biological information output unit, is equal to or greater than a predetermined similarity degree (S104). The operation of the determination unit 50 branches depending on whether or not the similarity degree is equal to or greater than the predetermined similarity degree (S106), if the similarity degree is equal to or greater than the predetermined similarity degree, the operation of the determination unit 50 proceeds to S108, and if the similarity degree is less than the predetermined similarity degree, the operation of the determination unit 50 proceeds to S110.

If the similarity degree is equal to or greater than the predetermined similarity degree, the determination unit 50 outputs the determination signal indicating the first determination result to the control unit 40 of the biological information output unit 200 (S108). On the other hand, if the similarity degree is less than the predetermined similarity degree, the determination unit 50 outputs the determination signal indicating the second determination result to the control unit 40 of the biological information output unit 200 (S110). By performing these determination operations, the operation of the determination unit 50 ends. The determination unit 50 may periodically and repeatedly perform the similarity degree determination. By such repetitive determination, the determination unit 50 may set, to a more suitable mode, the operations of the biological information output unit 100 and the biological information output unit 200 based on the correlation of noise and gradually transition to an optimum mode.

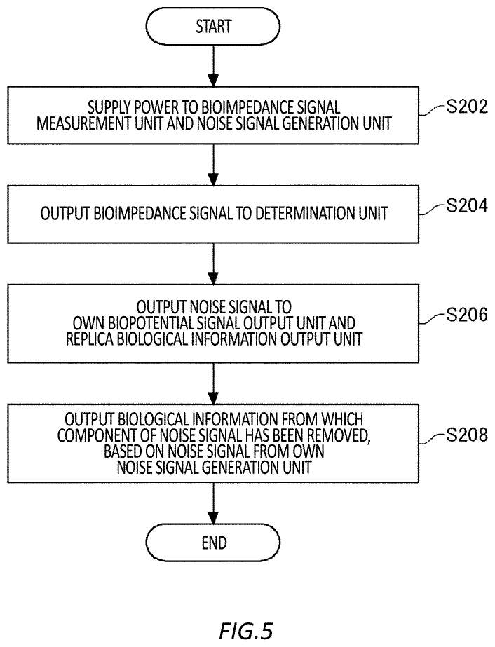

FIG. 5 is a flowchart illustrating an example of the operation of the biological information output unit 100 that is the main biological information output unit. The operation of the biological information output unit 100 of the present embodiment includes steps S202 to S208.

The biological information output unit 100 supplies power to the bioimpedance signal measurement unit 20 and the noise signal generation unit 30 (S202). The bioimpedance signal measurement unit 20 outputs the bioimpedance signal to the determination unit 50 (S204). Accordingly, the determination unit 50 can execute determination based on the bioimpedance signal and output a signal for deciding the amplification factor of the amplification unit 48 of the biological information output unit 200 that is the replica biological information output unit.

Next, when generating a noise signal indicating the influence of the MA based on the bioimpedance signal, the noise signal generation unit 30 outputs the generated noise signal to the biopotential signal output unit 12 of the corresponding biological information output unit 100 and the control unit 40 of the biological information output unit 200 (S206). Next, the biopotential signal output unit 12 outputs, as a signal indicating the biological information, the biopotential signal from which the component of the noise signal has been removed based on the noise signal output by the noise signal generation unit 30 (S208). After step S208, the operation of the biological information output unit 100 ends.

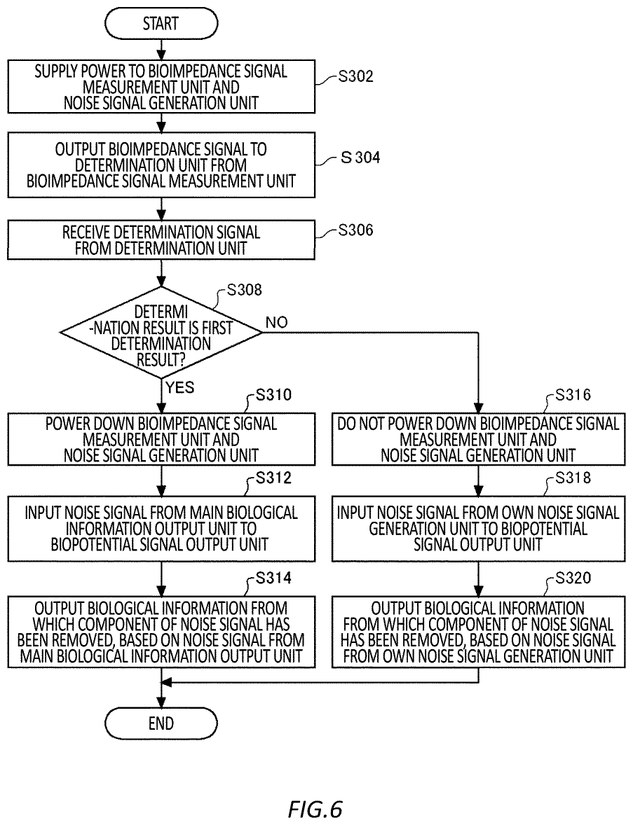

FIG. 6 is a flowchart illustrating an example of the operation of the biological information output unit 200 that is the replica biological information output unit. The operation of the biological information output unit 200 of the present embodiment includes steps S302 to S320. The biological information output unit 200 supplies power to the bioimpedance signal

measurement unit 20 and the noise signal generation unit 30 (S302). The bioimpedance signal measurement unit 20 outputs the bioimpedance signal to the determination unit 50 in order to perform determination based on the bioimpedance signals of the biological information output unit 100 and the biological information output unit 200 (S304). Next, the control unit 40 receives the determination signal from the determination unit 50 (S306). The operation of the biological information output unit 200 branches based on the determination result indicated by the determination signal (S308). In the control unit 40, in a case where the determination result indicated by the determination signal is the first determination result, the operation of the biological information output unit 200 proceeds to S310, and in a case where the similarity degree is less than the predetermined similarity degree, the operation of the biological information output unit 200 proceeds to S316.

If the determination signal received from the determination unit 50 indicates the first determination result, the control unit 40 powers down the bioimpedance signal measurement unit 20 and the noise signal generation unit 30 (S310). Accordingly, the power consumption is reduced in the biological information output unit 200. Next, the control unit 40 inputs, to the biopotential signal output unit, the noise signal from the biological information output unit 100A that is the main biological information output unit (S312). Based on the noise signal from the biological information output unit 100A that is the main biological information output unit, the biopotential signal output unit 212 outputs, as the signal indicating the biological information, the biopotential signal from which the component of the noise signal has been removed (S314).

If the determination signal received from the determination unit 50 indicates the first determination result, the bioimpedance signal measurement unit 20 and the noise signal generation unit 30 are not powered down (S316). Next, the control unit 40 inputs, to the biopotential signal output unit 212, the noise signal output by the noise signal generation unit 30 of the corresponding biological information output unit 200 (S318). The biopotential signal output unit 212 outputs, as the signal indicating the biological information, the biopotential signal from which the component of the noise signal has been removed based on the noise signal from the noise signal generation unit 30 of the corresponding biological information output unit 200 (S320).

After each of steps S314 and S320, the operation of the biological information output unit 200 ends. In a case where the determination unit 50 periodically and repeatedly performs the similarity degree determination, the control unit 40 may also periodically and repeatedly receive the determination signal. The control unit 40 may control the operation of the biological information output unit 200 to a more suitable mode based on the repeatedly received determination signals, and may gradually transition the operation of the biological information output unit 200 to an optimum mode.

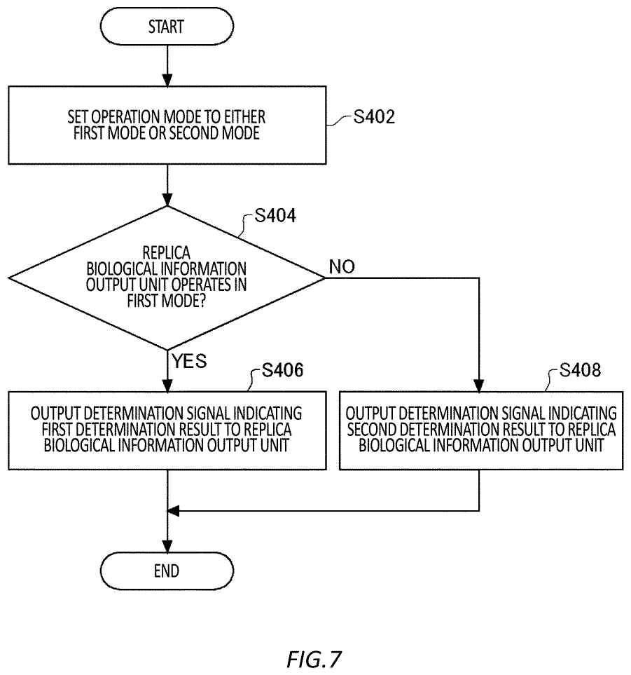

FIG. 7 is a flowchart illustrating an example of the operation of the determination unit 50 different from that in FIG. 4. The operation of the determination unit 50 of the present embodiment includes steps S402 to S408.

The determination unit 50 sets, to either a first mode or a second mode, the operation mode based on the reliability required for the biological information output from the biological information output unit 200 that is the replica biological information output unit (S402). The determination unit 50 determines whether the biological information output unit 200 is operating in the first mode or the second mode (S404). If the biological information output unit 200 is operating in the first mode, the determination unit 50 outputs the determination signal indicating the first determination result to the biological information output unit 200 (S406). On the other hand, if the biological information output unit 200 is operating in the second mode, the determination unit 50 outputs the determination signal indicating the second determination result to the biological information output unit 200 (S408). By performing these determination operations, the operation of the determination unit 50 ends.

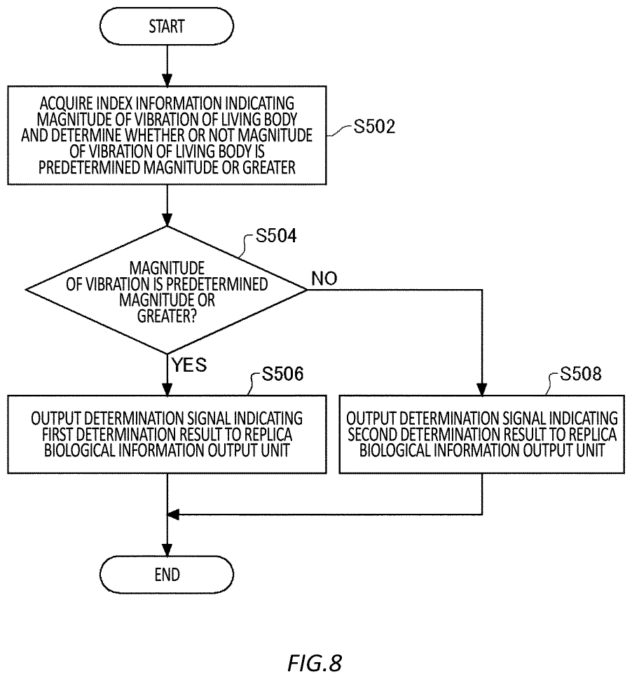

FIG. 8 is a flowchart illustrating an example of the operation of the determination unit 50 different from those in FIGS. 4 and 7. The operation of the determination unit 50 of the present embodiment includes steps S502 to S508.

The index information acquisition unit 54 of the determination unit 50 acquires index information indicating the magnitude of vibration of the living body 500. The determination unit 50 determines whether or not the magnitude of vibration of the living body 500 is equal to or greater than a predetermined magnitude based on the acquired index information (S502). The operation branches depending on whether or not the vibration is equal to or greater

than the predetermined magnitude (S504), if the vibration is equal to or greater than the predetermined magnitude, the operation of the determination unit 50 proceeds to S506, and if the similarity degree is less than the predetermined magnitude, the operation of the determination unit 50 proceeds to S508. If the vibration is equal to or greater than the predetermined magnitude, the determination unit 50 outputs the determination signal indicating the first determination result to the control unit 40 of the biological information output unit 200 (S506). On the other hand, if the similarity degree is less than the predetermined similarity degree, the determination unit 50 outputs the determination signal indicating the second determination result to the control unit 40 of the biological information output unit 200 (S508). By performing these determination operations, the operation of the determination unit 50 ends. The index information acquisition unit 54 may periodically and repeatedly determine the magnitude of vibration of the living body 500. By such repetition determination, the index information acquisition unit 54 may set, to a more suitable mode, the operations of the biological information output unit 100 and the biological information output unit 200 based on the magnitude of vibration of the living body 500 and gradually transition to an optimum mode.

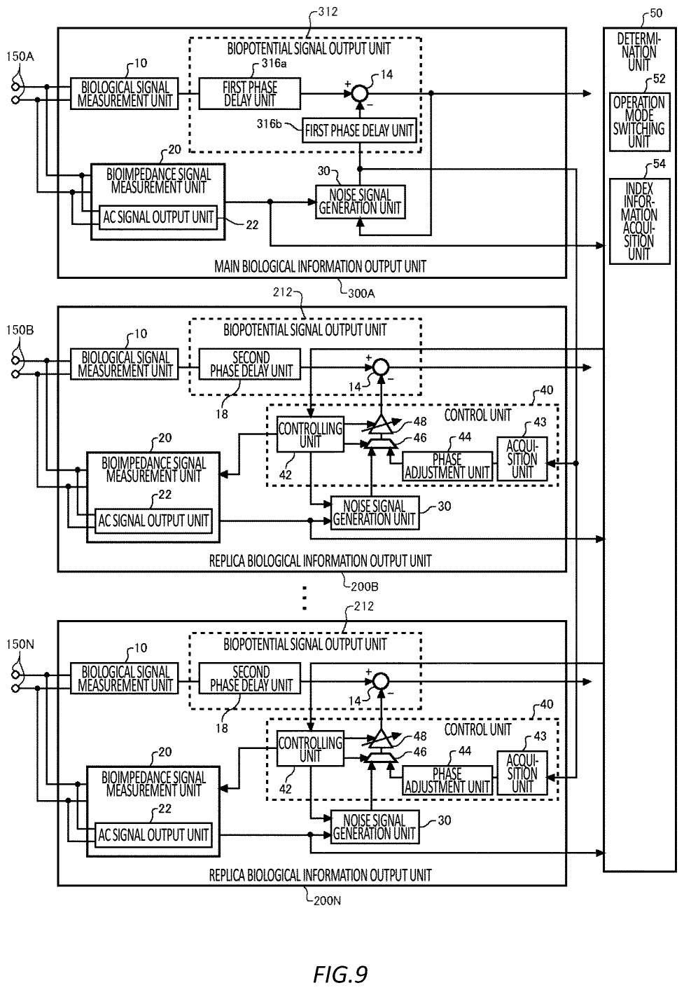

FIG. 9 illustrates an example of a block diagram of a configuration of a biological information providing apparatus in which a configuration of a biological information output unit 300 that is the main biological information output unit is different from that of the biological information output unit 100. Hereinafter, a description will be given focusing on a difference from the configuration of the biological information providing apparatus in FIG. 3.

The biological information providing apparatus includes the biological information output unit 300 that is the main biological information output unit, the biological information output units 200B to 200N that are the replica biological information output units, and the determination unit 50. The biological information providing apparatus in the drawing differs from the biological information providing apparatus in FIG. 3 only in the configuration of the biological information output unit 300 that is the main biological information output unit.

The biological information output unit 300 includes the biological signal measurement unit 10, the bioimpedance signal measurement unit 20, the noise signal generation unit 30, and a biopotential signal output unit 312. The biopotential signal output unit 312 includes a first phase delay unit 316a and a first phase delay unit 316b.

The first phase delay unit 316a and the first phase delay unit 316b are provided at a preceding stage (input side) of the subtractor 14, unlike the first phase delay unit 16 of the biological information output unit 100. Accordingly, the first phase delay unit 316a and the first phase delay unit 316b delay the phase at the preceding stage of inputting, to the subtractor 14, the biological signal output from the biological signal measurement unit 10 and the noise signal output from the noise signal generation unit 30.

Even in a case where the first phase delay unit 316a and the first phase delay unit 316b are provided at the preceding stage of the subtractor 14, the first phase delay unit 316a and the first phase delay unit 316b can adjust the phases of the biopotential signal output from a biological information output unit 300A and the biopotential signal output from the biological information output unit 200. By using the first phase delay unit 316a and the first phase delay unit 316b, the phase difference in the output signal between the different ICs is compensated, and the accuracy in performing gesture recognition using the myoelectric signal is improved.

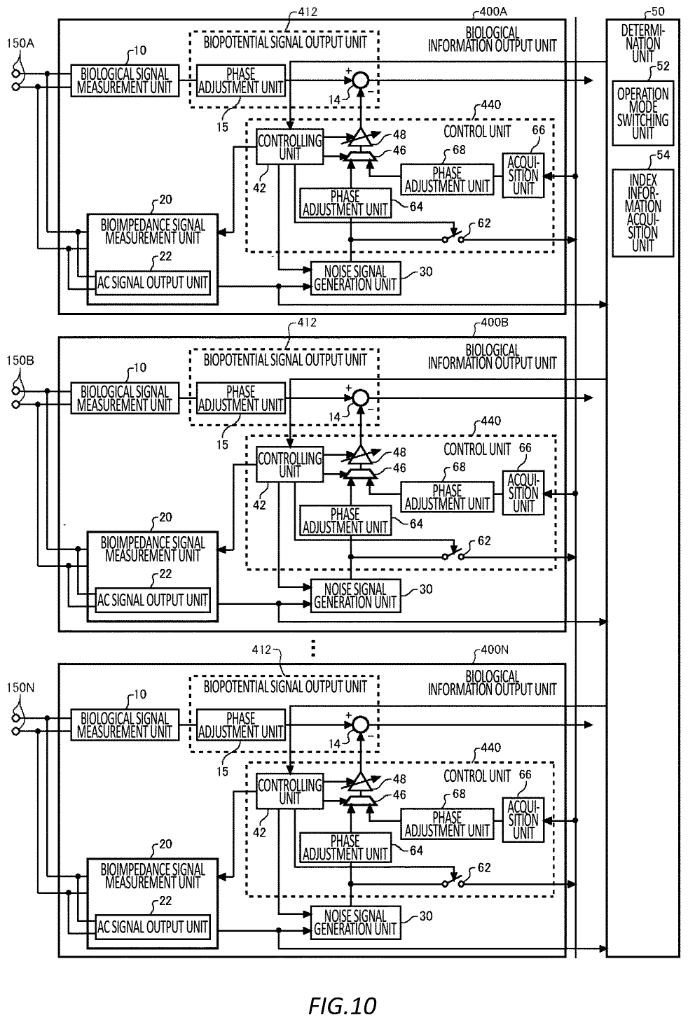

FIG. 10 illustrates an example of a block diagram of a biological information providing apparatus including a biological information output unit 400. The biological information providing apparatus of the present embodiment includes the biological information output unit 400 and the determination unit 50.

In the biological information output unit 400 in the present embodiment, whether to operate as the main biological information output unit or the replica biological information output unit is not determined in advance, and the role of the biological information output unit 400 dynamically changes based on the determination of the determination unit 50. The main biological information output unit and the replica biological information output unit belong to groups having similar MA components, and one main biological information output unit is set in each group. The biological information output unit 400 includes the biological signal measurement unit 10, the bioimpedance signal measurement unit 20, the noise signal generation unit 30, a control unit 440, and a biopotential signal output unit 412.

The noise signal generation unit 30 and the control unit 440 may be connected to a bus for transmitting and receiving the noise signal. Therefore, one replica biological information output unit of a plurality of replica biological information output units is associated with each of the main biological information output units via the bus. In the present embodiment, in a case where the determination signal indicates the first determination result, the control unit 440 of each replica biological information output unit performs control such that the noise signal generated by the noise signal generation unit 30 of the main biological information output unit associated with the corresponding replica biological information output unit is input to its own biopotential signal output unit 412.

The control unit 440 controls the noise signal input to the biopotential signal output unit 412, based on a signal indicating the identification information received from the determination unit 50. In a case where the biological information output unit 400 provided with the corresponding control unit 440 is the main biological information output unit, the control unit 440 performs control to output the noise signal output by the noise signal generation unit 30 of the biological information output unit 400 to the biopotential signal output unit 412 and the bus. On the other hand, in a case where the biological information output unit 400 provided with the corresponding control unit 440 is the replica biological information output unit, the control unit 440 acquires, from the bus, the noise signal output from the main biological information output unit of the group to which the corresponding biological information output unit 400 belongs. Next, the control unit 440 performs control to output the noise signal of the main biological information output unit to the biopotential signal output unit 412.

Therefore, in a plurality of biological information output units 400, under the control of the control unit 440, in a case where the corresponding biological information output unit 400 is not the main biological information output unit indicated by the identification information, the noise signal generated by the noise signal generation unit 30 of the main biological information output unit indicated by the identification information is input to its own biopotential signal output unit 412. On the other hand, in a case where the corresponding biological information output unit 400 is the main biological information output unit indicated by the identification information, the noise signal generated by its own noise signal generation unit 30 is input to its own biopotential signal output unit 412. The control unit 440 includes the controlling unit 42, the selector 46, the amplification unit 48, a switch 62, a phase adjustment unit 64, an acquisition unit 66, and a phase adjustment unit 68.

In the present embodiment, the determination signal received by the controlling unit 42 from the determination unit 50 includes identification information indicating whether the biological information output unit 400 is the main biological information output unit or the replica biological information output unit. The controlling unit 42 controls the biological information output unit 400 to operate as the main biological information output unit or the replica biological information output unit, based on the identification information.

The switch 62 is a switch that switches whether or not the noise signal generation unit 30 outputs the noise signal to the bus. The switch 62 is switched on and off by the controlling unit 42, and is switched on in a case where the biological information output unit 400 is the main biological information output unit, and is switched off in a case where the biological information output unit 400 is the replica biological information output unit.

The phase adjustment unit 64 has a configuration corresponding to that of the first phase delay unit 316b in the embodiment of FIG. 9. Phase adjustment similar to that of the first phase delay units 316a and 316b in the embodiment of FIG. 9 is performed by a combination of a phase adjustment unit 15 and the phase adjustment unit 64.

In a case where the biological information output unit 400 is the replica biological information output unit, the acquisition unit 66 acquires, from the bus, the noise signal from the main biological information output unit of the group to which the corresponding biological information output unit 400 belongs. The noise signal may be attached with information that allows for identification of the main biological information output unit having output the noise signal. Therefore, the acquisition unit 66 can selectively acquire the noise signal of the main biological information output unit of the group to which the corresponding biological information output unit 400 belongs. The acquisition unit 66 has a configuration corresponding to that of the acquisition unit 43 of the embodiment of FIGS. 3 and 9.

The phase adjustment unit 68 has a configuration corresponding to that of the phase adjustment unit 44 in the embodiment of FIG. 9. Therefore, between the ICs of the main biological information output unit and the replica biological information output unit, the phase adjustment unit 68 performs timing compensation associated with clock system switching when the data synchronized with the main biological information output unit is passed to the replica biological information output unit.

In a case where the biological information output unit 400 is the main biological information output unit, the selector 46 outputs, to the amplification unit 48, a signal obtained by the phase adjustment unit 64 phase-adjusting the noise signal output from the noise signal generation unit 30 of the corresponding biological information output unit 400. On the other hand, in a case where the biological information output unit 400 is the replica biological information output unit, a signal obtained by the phase adjustment unit 68 phase-adjusting the noise signal output from the main biological information output unit is output to the amplification unit 48.

The amplification unit 48 amplifies the signal to be output to the biopotential signal output unit 412, based on the amplification factor adjusted by the controlling unit 42. A signal based on the level ratio of the amplitude of the bioimpedance signals of the main biological information output unit and the replica biological information output unit is input from the determination unit 50 to the controlling unit 42. In particular, in a case where the biological information output unit 400 is the replica biological information output unit, the controlling unit 42 adjusts the amplification factor of the amplification unit 48, based on the level ratio.

The biopotential signal output unit 412 phase-adjusts the biological signal output from the biological signal measurement unit 10, removes the noise signal component proportional to the MA from the biological signal, and outputs a biopotential signal indicating biological information. The biopotential signal output unit 412 includes the phase adjustment unit 15 and the subtractor 14.

In a case where the biological information output unit 400 is the main biological information output unit, the phase adjustment unit 15 functions as a configuration similar to that of the first phase delay unit 316a in the embodiment of FIG. 9. In a case where the biological information output unit 400 is the replica biological information output unit, the phase adjustment unit 15 functions as a configuration similar to that of the second phase delay unit 18 in the embodiment of FIG. 9.

The subtractor 14 subtracts the signal output by the amplification unit 48 from the phase-adjusted biological signal output by the phase adjustment unit 15. Accordingly, the biopotential signal output unit 412 can output, as the signal indicating the biological information, the bioelectronic signal obtained by removing the influence of the MA from the biological signal.

The determination unit 50 of the present embodiment outputs identification information for identifying the main biological information output unit among the plurality of biological information output units 400, based on a predetermined condition.

As the predetermined condition, the determination unit 50 may output the identification information indicating the main biological information output unit and the replica biological information output unit, based on the similarity degree of the bioimpedance signals. The determination unit 50 may group the biological information output units 400 based on the similarity degree of the bioimpedance signals of the biological information output units 400 and output identification information for identifying the main biological information output unit for each group. Alternatively, the determination unit 50 may group the biological information output units 400 based on the similarity degree between the noise signals generated by the respective noise signal generation units 30 of the biological information output units 400, and output the identification information for identifying the main biological information output unit for each group. Furthermore, the determination unit 50 may output the identification information indicating the main biological information output unit and the replica biological information output unit, based on the similarity degree of the signals of the biopotential signal output units.

As the predetermined condition, the determination unit 50 may output the identification information indicating the main biological information output unit and the replica biological information output unit, based on a condition corresponding to the operation mode based on the reliability required for the biological information. The biological information providing apparatus may be operable in the first mode in which the biological information output unit 400 outputs the biopotential signal with the first reliability and the second mode in which the biological information output unit 400 outputs the biopotential signal with the second reliability higher than the first reliability. In this case, in a case where the biological information providing apparatus operates in the first mode, the determination unit 50 may output, to each of the biological information output units 400, the identification information for identifying the main biological information output unit and the replica biological information output unit. On the other hand, in a case where the biological information providing apparatus operates in the second mode, the control unit 440 may perform control such that the noise signal generated by the noise signal generation unit 30 of the corresponding biological information output unit 400 is input to its own biopotential signal output unit 412.

As another example, the biological information providing apparatus may be operable in the first mode in which the biopotential signal is output with the first reliability, the second mode in which the biopotential signal is output with the second reliability higher than the first reliability, and a third mode in which the biopotential signal is output with third reliability degree higher than the second reliability. The plurality of biological information output units 400 may be grouped into a first number of first groups and may also be grouped into a second number of second groups, the second number being larger than the first number. In this case, in a case where the biological information providing apparatus operates in the first mode, the determination unit 50 may output, to each of the biological information output units 400, the identification information indicating the main biological information output unit for each first group. On the other hand, in a case where the biological information providing apparatus operates in the second mode, the determination unit 50 may output, to each of the biological information output units 400, the identification information indicating the main biological information output unit for each second group. In a case where the biological information providing apparatus operates in the third mode, the control unit 440 may perform control such that the noise signal generated by the noise signal generation unit 30 of the corresponding biological information output unit 400 is input to the biopotential signal output unit 412 of the corresponding biological information output unit 400.

Here, a case will be described in which the plurality of biological information output units 400 are grouped into the first number of first groups in the first mode when the biological information providing apparatus outputs the biopotential signal with the first reliability. Among the first reliability, the second reliability, and the third reliability, the first reliability is the lowest reliability. As described above, there is a trade-off relationship between increasing the number of main biological information output units (increasing the number of groups) to increase the reliability of the biological information and reducing the power consumption by causing a larger number of replica biological information output units to use the noise signal of the main biological information output unit. Therefore, in the case of the first reliability, the biological information output units 400 are grouped into the smallest number of groups. In this case, the number of main biological information output units that output the noise signals generated by themselves is reduced, and the power consumption is reduced the most.

In the second mode in which the biological information output units 400 are grouped into the second number of groups, the number of main biological information output units that use the noise signals generated by their own noise signal generation units 30 is larger than that in a case where the biological information output units 400 are classified into the first number of groups. Therefore, while the reliability with respect to the noise removal is improved, the power consumption is greater than that in the case of being grouped into the first number of groups.

In the third mode, the noise signal generated by the corresponding biological information output unit 400 is input to the biological information output unit 400. This indicates that the biological information output unit 400 is operated as the main biological information output unit. In this case, while the reliability of the operation of removing noise from the biological information increases, the power consumption of the biological information output unit 400 increases.

Although three operation modes are mentioned in the present embodiment, the number of operation modes is not limited to three. In other words, the biological information providing apparatus may have four or more operation modes according to the reliability. By switching the operation mode according to the reliability required for the biological information, the biological information providing apparatus can implement the power consumption and the reliability in a desired balance.

In the present embodiment, the determination unit 50 functions as an “identification information output unit” that outputs the identification information for identifying the biological information output unit 400 as the main biological information output unit or the replica biological information output unit. In addition, the main biological information output unit corresponds to a “specific biological information output unit” in a group to which one or more biological information output units 400 belong.

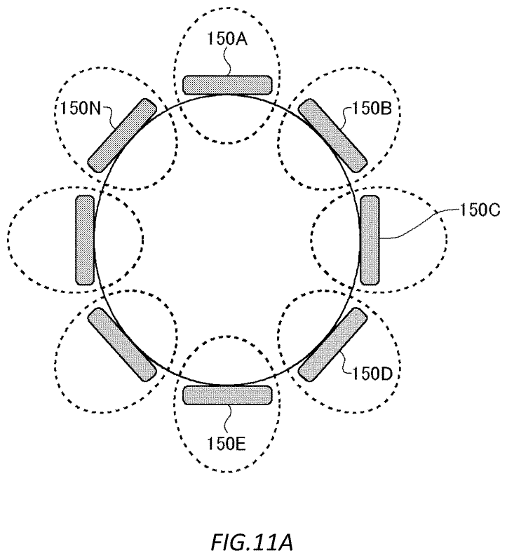

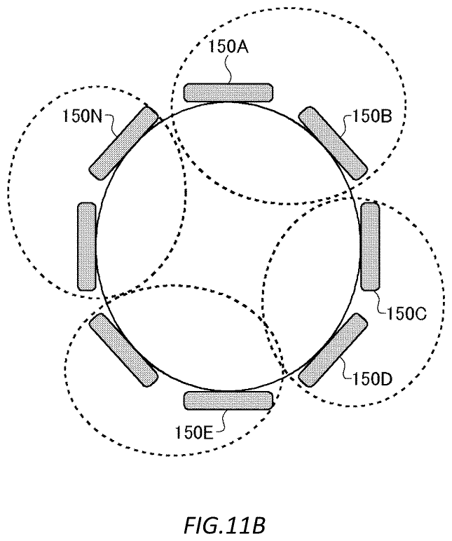

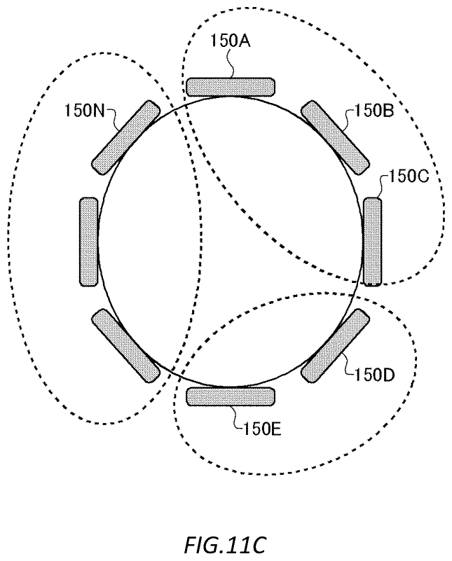

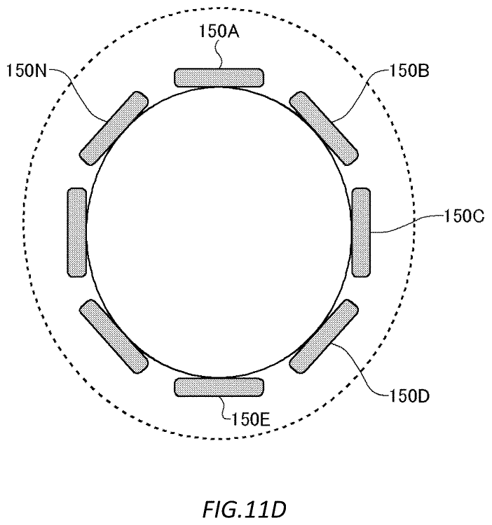

FIG. 11A illustrates an example of grouping of the electrodes 150A to 150N connected to the biological information output units 400. In the drawing, the biological information output units 400A to 400N connected to the N pairs of electrodes 150 are classified into groups each including one biological information output unit.

In each group, one biological information output unit 400 is classified as the main biological information output unit. In the present embodiment, since each group includes one biological information output unit 400, all the biological information output units 400 are classified as the main biological information output units. Therefore, each biological information output unit 400 outputs the biopotential signal indicating the biological information from which the influence of the MA has been removed, based on the noise signal output by its own noise signal generation unit 30.

FIG. 11B illustrates an example of grouping of the electrodes 150A to 150N connected to the biological information output units 400. In the present embodiment, the biological information output units 400A to 400N connected to the N pairs of electrodes 150 are classified into groups each including two biological information output units.

In this example, since the MAs measured at adjacent electrodes are likely to be similar, the adjacent electrodes 150 are paired. Since each group includes one main biological information output unit, for example, in a case where N is an even number, N/2 biological information output units 400 are classified as the main biological information output units, and N/2 biological information output units 400 are classified as the replica biological information output units. In this example, similarly, for example, in a case where N is an odd number, (N+1)/2 is classified as the main biological information output units, and (N−1)/2 is classified as the replica biological information output units.

The biological information output unit 400 classified as the replica biological information output unit acquires the noise signal from the main biological information output unit belonging to the group to which the corresponding biological information output unit 400 belongs. The biological information output unit 400 classified as the replica biological information output unit outputs the biopotential signal indicating the biological information from which the influence of the MA has been removed, based on the noise signal of the main biological information output unit. FIG. 11C illustrates an example of grouping of the electrodes 150A to 150N connected to the biological information output units 400. In the example of FIG. 11A, the number of replica biological information output units included in each group is 0, and in the example of FIG. 11B, the number of replica biological information output units included in each group is 1. Therefore, in any of the examples of FIGS. 11A and 11B, the number of replica biological information output units included in each group is the same.

However, the number of replica biological information output units included in each group may differ. In the embodiment of FIG. 11C, the number of replica biological information output units included in each group differs. In the drawing, the electrode 150A, the electrode 150B, and the electrode 150C are included in one group, any one of the biological information output unit 400A, the biological information output unit 400B, and the biological information output unit 400C is classified as the main biological information output unit, and the other two are classified as the replica biological information output units. Therefore, the number of replica biological information output units included in this group is two.

Next, in the group including the electrode 150D and the electrode 150E, either the biological information output unit 400D or the biological information output unit 400E is classified as the main biological information output unit, and the other of the biological information output unit 400D and the biological information output unit 400E is classified as the replica biological information output unit. Therefore, the number of replica biological information output units included in this group is one.

As described above, the number of replica biological information output units is different between the group including the electrodes 150A, 150B, and 150C and the group including the electrodes 150D and 150E. The classification of the main biological information output unit and the replica biological information output unit may be based on the similarity degree of the bioimpedance signals. The determination unit 50 groups the biological information output units 400 in a case where grouping is possible such that the similarity degree is equal to or greater than a predetermined similarity degree. The fact that the number of replica devices included in the group differs indicates that, in this case, the number of biological information output units 400 included in one group may not be specified in advance.

FIG. 11D illustrates an example of grouping of the electrodes 150A to 150N connected to the biological information output units 400. In the drawing, the biological information output units 400A to 400N connected to the N pairs of electrodes 150 are classified into one group including all the biological information output units.

In the present embodiment, any one of the biological information output units 400A to 400N (for example, the biological information output unit 400A) operates as the main biological information output unit. In this case, which of the biological information output units 400A to 400N operates as the main biological information output unit may be determined based on the bioimpedance signal output from the bioimpedance signal measurement unit 20. For example, in a case where the biological information output unit 400 is selected as the main biological information output unit, the biological information output unit 400 may be determined based on the sum of the squares of the quantified differences in the bioimpedance signal between the main and the replicas, with the sum minimized. For example, in a case where the biological information output unit 400A operates as the main biological information output unit, the remaining N−1 biological information output units 400, that is, the biological information output units 400B to 400N operate as the replica biological information output units.

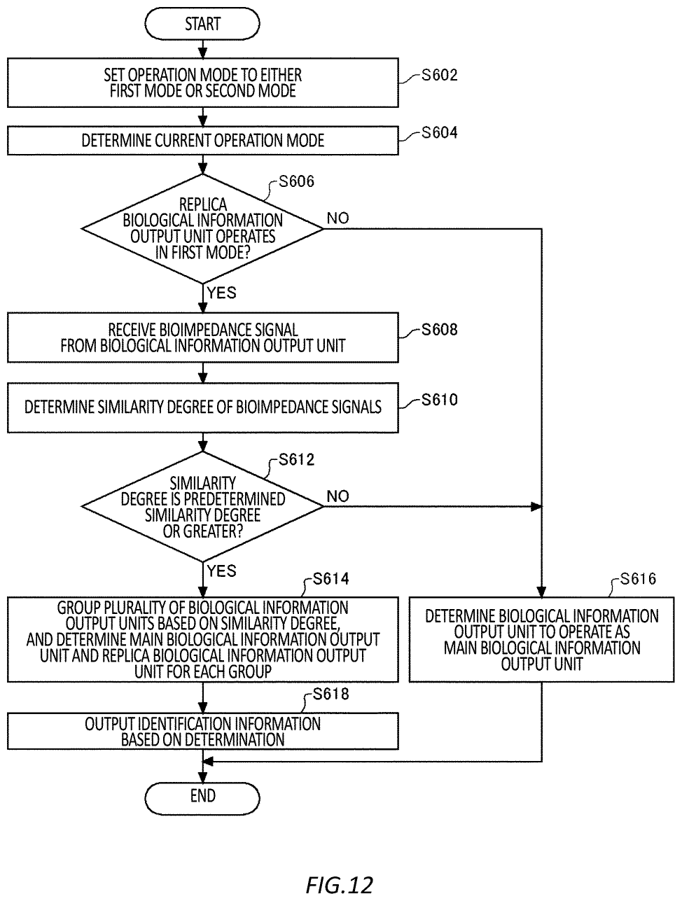

FIG. 12 is a flowchart illustrating an example of the operation of the determination unit 50 in the embodiment of FIG. 10. The operation of the determination unit 50 of the present embodiment includes steps S602 to S618.

The determination unit 50 sets the operation mode of the biological information providing apparatus to either the first mode or the second mode (S602). The determination unit 50 determines a current operation mode in which the replica biological information output unit operates (S604). The operation branches depending on whether the current operation mode in which the replica biological information output unit operates is the first operation mode or the second operation mode (S606), if the current operation mode is the first operation mode, the operation of the determination unit 50 proceeds to S608, and if the current operation mode is the second operation mode, the operation of the determination unit 50 proceeds to S616.

If the current operation mode is the first operation mode, the determination unit 50 receives the bioimpedance signal from the bioimpedance signal measurement unit 20 of each of the biological information output units 400 (S608). Next, the determination unit 50 determines the similarity degree of the bioimpedance signals (S610). The operation of the determination unit 50 branches depending on whether or not the similarity degree is equal to or greater than the predetermined similarity degree (S612), if the similarity degree is equal to or greater than the predetermined similarity degree, the operation of the determination unit 50 proceeds to S614, and if the similarity degree is less than the predetermined similarity degree, the operation of the determination unit 50 proceeds to S616.

If the similarity degree is equal to or greater than the predetermined similarity degree, the plurality of biological information output units 400 are grouped based on the similarity degree, and the main biological information output unit and the replica biological information output unit are determined for each group (S614). On the other hand, if the similarity degree is less than the predetermined similarity degree, the determination unit 50 determines each of the biological information output units 400 to operate as the main biological information output unit (S616). Based on the determination in S614 or S616, the determination unit 50 outputs the identification information to the biological information output unit 400 (S618). By performing these determination operations, the operation of the determination unit 50 ends.

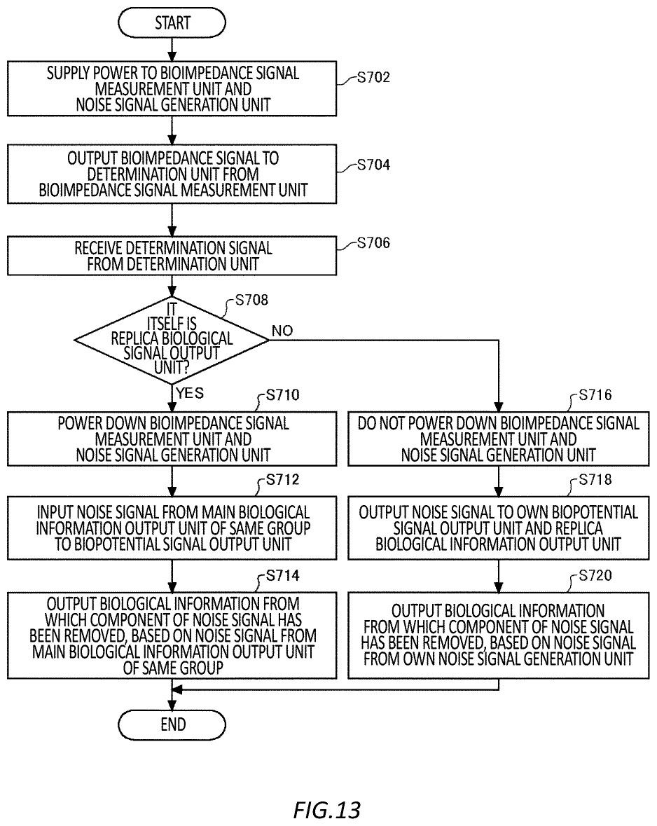

FIG. 13 is a flowchart illustrating an example of the operation of the biological information output unit 400 in the embodiment of FIG. 10. The operation of the biological information output unit 400 of the present embodiment includes steps S702 to S720.

The biological information output unit 400 supplies power to the bioimpedance signal measurement unit 20 and the noise signal generation unit 30 (S702). The bioimpedance signal measurement unit 20 outputs the bioimpedance signal to the determination unit 50 in order to determine the similarity degree of the bioimpedance signals (S704). The control unit 440 of the biological information output unit 400 receives the identification information from the determination unit 50 (S706). The operation of the biological information output unit 400 branches depending on whether or not the information indicated by the identification information indicates the corresponding biological information output unit 400 is the replica biological signal output unit (S708). If the information indicated by the identification information indicates that the corresponding biological information output unit 400 is the replica biological signal output unit, the operation of the biological information output unit 400 proceeds to S710. If the information indicated by the identification information does not indicate that the corresponding biological information output unit 400 is the replica biological signal output unit, the operation of the biological information output unit 400 proceeds to S716.

If the information indicated by the identification information indicates that the corresponding biological information output unit 400 is the replica biological signal output unit, the control unit 440 powers down the bioimpedance signal measurement unit and the noise signal generation unit (S710). The control unit 440 inputs the noise signal from the bioimpedance signal measurement unit 20 of the main biological information output unit of the same group to its own biopotential signal output unit 412 (S712). The biological information output unit 400 that is the replica biological signal output unit outputs the biopotential signal indicating the biological information from which the component of the noise signal has been removed, based on the noise signal from the main biological information output unit of the same group (S714).

If the information indicated by the identification information does not indicate that the corresponding biological information output unit 400 is the replica biological signal output unit, that is, if it is indicated that the corresponding biological information output unit 400 is the main biological information output unit, the control unit 440 does not power down the bioimpedance signal measurement unit 20 and the noise signal generation unit 30 (S716). The control unit 440 outputs the noise signal to the biopotential signal output unit 412 of the corresponding biological information output unit 400 and another biological information output unit 400 that is the replica biological information output unit belonging to the same group (S718). The biological information output unit 400 that is the main biological information output unit outputs the biopotential signal indicating the biological information from which the component of the noise signal has been removed, based on the noise signal from its own noise signal generation unit 30 (S720). After each of steps S714 and S720, the operation of the biological information output unit 200 ends.

As described above, according to the biological information providing apparatus of the present embodiment, the power consumption of the biological information providing apparatus can be suppressed by appropriately sharing the noise signal used in each biological information output unit according to the predetermined condition.

While the present invention has been described by way of the embodiments, the technical scope of the present invention is not limited to the scope described in the above-described embodiments. It is apparent to persons skilled in the art that various alterations or improvements can be added to the above-described embodiments. It is also apparent from the scope of the claims that the embodiments added with such alterations or improvements can be included in the technical scope of the invention.

Note that the order of execution of each process such as operations, procedures, steps, stages in the apparatus, system, program, and method shown in the claims, specification, and diagrams can be realized in any order as long as the order is not specifically indicated by “prior to,” “before,” or the like and also as long as the output from a previous process is not used in a later process. Even if the operational flow is described by using phrases such as “first” or “next” in the claims, specification, or diagrams for convenience, it does not necessarily mean that the process must be performed in this order.

### Other Possible Items

### Item 1

A biological information providing apparatus including:
a first biological information output unit and a second biological information output unit each including
   a biological signal measurement unit which measures a biological signal through a pair of electrodes in contact with a same living body,
   a bioimpedance signal measurement unit which measures a bioimpedance generated between the pair of electrodes to output a bioimpedance signal corresponding to the bioimpedance,
   a noise signal generation unit which generates, from the bioimpedance signal, a noise signal indicating a noise component included in the biological signal, and
   a biopotential signal output unit which removes a component of the noise signal from the biological signal to output a resultant signal as a biopotential signal; and
a determination unit which outputs a determination signal based on a predetermined condition, wherein
the second biological information output unit further includes
a control unit which performs control such that in a case where the determination signal indicates a first determination result, the noise signal generated by the noise signal generation unit of the first biological information output unit is input to the biopotential signal output unit of the second biological information output unit, and
in a case where the determination signal indicates a second determination result, the noise signal generated by the noise signal generation unit of the second biological information output unit is input to the biopotential signal output unit of the second biological information output unit.

### Item 2

The biological information providing apparatus according to item 1, wherein
the noise signal generation unit includes
an adaptive filter which generates the noise signal based on the bioimpedance signal and a coefficient, and
the biopotential signal output unit
adds the biological signal and the noise signal to output a resultant signal as the biopotential signal.

### Item 3

The biological information providing apparatus according to item 1, wherein in the case where the determination signal indicates the first determination result, the control unit controls at least one of the bioimpedance signal measurement unit or the noise signal generation unit of the second biological information output unit so as to suppress power consumption in at least one of the bioimpedance signal measurement unit or the noise signal generation unit of the second biological information output unit.

### Item 4

The biological information providing apparatus according to item 3, wherein the control unit performs control to suppress the power consumption by executing at least one of stopping supply of the bioimpedance signal by the bioimpedance signal measurement unit or stopping generation of the noise signal by the noise signal generation unit.

### Item 5

The biological information providing apparatus according to item 4, wherein
the bioimpedance signal measurement unit includes an AC signal supply unit which supplies an AC signal between a pair of electrodes in contact with the living body, and
the control unit performs control to suppress the power consumption by stopping supply of the AC signal by the AC signal supply unit.

### Item 6

The biological information providing apparatus according to item 1, wherein
the bioimpedance signals of the first biological information output unit and the second biological information output unit or the biopotential signals of the first biological information output unit and the second biological information output unit are input to the determination unit, and
the determination unit outputs a determination signal based on the bioimpedance signals or the biopotential signals having been input.

### Item 7

The biological information providing apparatus according to item 1, wherein
the determination unit
outputs the determination signal indicating the first determination result according to the predetermined condition in a case where a similarity degree between the bioimpedance signal of the first biological information output unit and the bioimpedance signal of the second biological information output unit is equal to or greater than a predetermined similarity degree, and
outputs the determination signal indicating the second determination result according to the predetermined condition in a case where a similarity degree between the bioimpedance signal of the first biological information output unit and the bioimpedance signal of the second biological information output unit is less than the predetermined similarity degree.

### Item 8

The biological information providing apparatus according to item 1, wherein
the determination unit
outputs the determination signal indicating the first determination result according to the predetermined condition in a case where a similarity degree between the biopotential signal of the first biological information output unit and the biopotential signal of the second biological information output unit is equal to or greater than a predetermined similarity degree, and
outputs the determination signal indicating the second determination result according to the predetermined condition in a case where a similarity degree between the biopotential signal of the first biological information output unit and the biopotential signal of the second biological information output unit is less than the predetermined similarity degree.

### Item 9

The biological information providing apparatus according to item 1, wherein
the determination unit
acquires index information indicating a magnitude of vibration of the living body,
outputs the determination signal indicating the first determination result according to the predetermined condition in a case where the index information indicates that a magnitude of vibration of the living body is equal to or greater than a predetermined magnitude, and
outputs the determination signal indicating the second determination result according to the predetermined condition in a case where the index information indicates that a magnitude of vibration of the living body is less than the predetermined magnitude.

### Item 10

The biological information providing apparatus according to item 9, wherein
the determination unit
acquires, as the index information, at least one of position information of the living body, vibration information including the magnitude of vibration of the living body, or acceleration information of the living body.

### Item 11

The biological information providing apparatus according to item 1, wherein
the biological information providing apparatus is operable in a first mode in which the second biological information output unit outputs the biopotential signal with first reliability and a second mode in which the second biological information output unit outputs the biopotential signal with second reliability higher than the first reliability, and
the determination unit
outputs the determination signal indicating the first determination result according to the predetermined condition in a case where the biological information providing apparatus operates in the first mode, and
outputs the determination signal indicating the second determination result according to the predetermined condition in a case where the biological information providing apparatus operates in the second mode.

### Item 12

The biological information providing apparatus according to item 1, wherein the second biological information output unit includes an amplification unit which amplifies or attenuates the noise signal, which serves as an input to the biopotential signal output unit of the second biological information output unit, with an amplification factor based on a level ratio between the bioimpedance signal measured by the bioimpedance signal measurement unit of the first biological information output unit and the bioimpedance signal measured by the bioimpedance signal measurement unit of the second biological information output unit, and then outputs a resultant signal to the biopotential signal output unit of the second biological information output unit.

### Item 13

The biological information providing apparatus according to any one of items 1 to 12, wherein
the biological information providing apparatus includes a plurality of second biological information output units including the second biological information output unit, and
the determination unit outputs the determination signal to each of the plurality of second biological information output units according to the predetermined condition.

### Item 14

The biological information providing apparatus according to item 13, further including a band which holds the pair of electrodes of each of the first biological information output unit and the plurality of second biological information output units and is worn on the living body.

### Item 15

The biological information providing apparatus according to any one of items 1 to 12, wherein
the biological information providing apparatus includes a plurality of first biological information output units including the first biological information output unit and a plurality of second biological information output units including the second biological information output unit,
at least one second biological information output unit of the plurality of second biological information output units is associated with each of the plurality of first biological information output units, and
in the case where the determination signal indicates the first determination result, the control unit of each of the plurality of second biological information output units performs control such that the noise signal generated by the noise signal generation unit of the first biological information output unit associated with the corresponding second biological information output unit is input to the biopotential signal output unit of the corresponding second biological

### Item 16

The biological information providing apparatus according to item 15, further including a band which holds the pair of electrodes of each of the plurality of first biological information output units and the plurality of second biological information output units and is worn on the living body.

### Item 17

The biological information providing apparatus according to any one of items 1 to 12, wherein
the first biological information output unit further includes
a first phase delay unit which synchronizes the biopotential signal output by the biopotential signal output unit of the second biological information output unit with the biopotential signal output by the biopotential signal output unit of the corresponding first biological information output unit, and
the second biological information output unit further includes a second phase delay unit which synchronizes the biological signal with the noise signal generated by the noise signal generation unit of the first biological information output unit.

### Item 18

A biological information providing apparatus including:
a plurality of biological information output units each including
a biological signal measurement unit which measures a biological signal through a pair of electrodes in contact with a same living body,
a bioimpedance signal measurement unit which measures a bioimpedance generated between the pair of electrodes to output a bioimpedance signal corresponding to the bioimpedance,
a noise signal generation unit which generates, from the bioimpedance signal, a noise signal indicating a noise component included in the biological signal, and
a biopotential signal output unit which removes a component of the noise signal from the biological signal to output a resultant signal as a biopotential signal; and
an identification information output unit which groups the plurality of biological information output units based on a similarity degree of the bioimpedance signals of the plurality of biological information output units, and outputs identification information for identifying a specific biological information output unit for each group, wherein
the plurality of biological information output units each further include
a control unit which performs control such that in a case where the corresponding biological information output unit is not a specific biological information output unit indicated by the identification information, a noise signal generated by the noise signal generation unit of the specific biological information output unit indicated by the identification information is input to its own biopotential signal output unit, and
in a case where the corresponding biological information output unit is a specific biological information output unit indicated by the identification information, a noise signal generated by its own noise signal generation unit is input to its own biopotential signal output unit.

### Item 19

A biological information providing apparatus including:
a plurality of biological information output units each including
a biological signal measurement unit which measures a biological signal through a pair of electrodes in contact with a same living body,
a bioimpedance signal measurement unit which measures a bioimpedance generated between the pair of electrodes to output a bioimpedance signal corresponding to the bioimpedance,
a noise signal generation unit which generates, from the bioimpedance signal, a noise signal indicating a noise component included in the biological signal, and
a biopotential signal output unit which removes a component of the noise signal from the biological signal to output a resultant signal as a biopotential signal; and
an identification information output unit which outputs identification information for identifying a specific biological information output unit among the plurality of biological information output units based on a predetermined condition, wherein
the plurality of biological information output units each further include
a control unit which performs control such that in a case where the corresponding biological information output unit is not a specific biological information output unit indicated by the identification information, a noise signal generated by the noise signal generation unit of the specific biological information output unit indicated by the identification information is input to its own biopotential signal output unit, and
in a case where the corresponding biological information output unit is a specific biological information output unit indicated by the identification information, a noise signal generated by its own noise signal generation unit is input to its own biopotential signal output unit.

### Item 20

The biological information providing apparatus according to item 19, wherein
the biological information providing apparatus is operable in a first mode in which the plurality of biological information output units output the biopotential signal with first reliability and a second mode in which the plurality of biological information output units output the biopotential signal with second reliability higher than the first reliability,
in a case of operation in the first mode, the identification information output unit outputs the identification information to each of the plurality of biological information output units, and
in a case of operation in the second mode, the control unit performs control such that a noise signal generated by its own noise signal generation unit is input to its own biopotential signal output unit.

### Item 21

The biological information providing apparatus according to item 19, wherein
the biological information providing apparatus is operable in a first mode in which the plurality of biological information output units output the biopotential signal with first reliability, a second mode in which the plurality of biological information output units output the biopotential signal with second reliability higher than the first reliability, and a third mode in which the plurality of biological information output units output the biopotential signal with third reliability higher than the second reliability,
the plurality of biological information output units are grouped into a first number of first groups and is also grouped into a second number of second groups, the second number being larger than the first number,
the identification information output unit
outputs the identification information indicating a specific biological information output unit for each of the first groups to each of the plurality of biological information output units in a case where the biological information providing apparatus operates in the first mode, and
outputs the identification information indicating a specific biological information output unit for each of the second groups to each of the plurality of biological information output units in a case where the biological information providing apparatus operates in the second mode, and
in a case where the biological information providing apparatus operates in the third mode, the control unit performs control such that a noise signal generated by its own noise signal generation unit is input to its own biopotential signal output unit.

### Item 22

A biological information output circuit including:
a biological signal measurement unit which measures a biological signal through a pair of electrodes in contact with a same living body;
a bioimpedance signal measurement unit which measures a bioimpedance generated between the pair of electrodes to output a bioimpedance signal corresponding to the bioimpedance;
a noise signal generation unit which generates, from the bioimpedance signal, a noise signal indicating a noise component included in the biological signal;
a biopotential signal output unit which removes a component of the noise signal from the biological signal to output a resultant signal as a biopotential signal; and
a control unit which performs control such that in a case where a determination signal output by a determination unit based on a predetermined condition indicates a first determination result, an input external noise signal is input to the biopotential signal output unit, and
in a case where the determination signal indicates a second determination result, the noise signal generated by the noise signal generation unit is input to the biopotential signal output unit.

### EXPLANATION OF REFERENCES

10: biological signal measurement unit;
12: biopotential signal output unit;
14: subtractor;
16: first phase delay unit;
18: second phase delay unit;
20: bioimpedance signal measurement unit;
22: AC signal output unit;
30: noise signal generation unit;
40: control unit;
42: controlling unit;
43: acquisition unit;
44: phase adjustment unit;
46: selector;
48: amplification unit;
50: determination unit;
62: switch;
64: phase adjustment unit;
66: acquisition unit;
68: phase adjustment unit;
100: biological information output unit;
150: electrode;
200: biological information output unit;
212: biopotential signal output unit;
300: biological information output unit;
312: biopotential signal output unit;
316: first phase delay unit;
400: biological information output unit;
412: biopotential signal output unit;
440: control unit;
500: living body;
502: epidermal layer;
504: dermal and subcutaneous layers;
506: muscle layer;
508: muscle fiber;
510: nerve;
520: surface muscle; and
530: deep muscle.

What is claimed is:

1. A biological information providing apparatus comprising:
a first biological information output unit and a second biological information output unit each including
a biological signal measurement unit which measures a biological signal through a pair of electrodes in contact with a same living body,
a bioimpedance signal measurement unit which measures a bioimpedance generated between the pair of electrodes to output a bioimpedance signal corresponding to the bioimpedance,
a noise signal generation unit which generates, from the bioimpedance signal, a noise signal indicating a noise component included in the biological signal, and
a biopotential signal output unit which removes a component of the noise signal from the biological signal to output a resultant signal as a biopotential signal; and
a determination unit which outputs a determination signal based on a predetermined condition, wherein
the second biological information output unit further includes
a control unit which performs control such that in a case where the determination signal indicates a first determination result, the noise signal generated by the noise signal generation unit of the first biological information output unit is input to the biopotential signal output unit of the second biological information output unit, and
in a case where the determination signal indicates a second determination result, the noise signal generated by the noise signal generation unit of the second biological information output unit is input to the biopotential signal output unit of the second biological information output unit.

2. The biological information providing apparatus according to claim 1, wherein
the noise signal generation unit includes
an adaptive filter which generates the noise signal based on the bioimpedance signal and a coefficient, and
the biopotential signal output unit
adds the biological signal and the noise signal to output a resultant signal as the biopotential signal.

3. The biological information providing apparatus according to claim 1, wherein in the case where the determination signal indicates the first determination result, the control unit controls at least one of the bioimpedance signal measurement unit or the noise signal generation unit of the second biological information output unit so as to suppress power consumption in at least one of the bioimpedance signal measurement unit or the noise signal generation unit of the second biological information output unit.

4. The biological information providing apparatus according to claim 3, wherein the control unit performs control to suppress the power consumption by executing at least one of stopping supply of the bioimpedance signal by the bioimpedance signal measurement unit or stopping generation of the noise signal by the noise signal generation unit.

5. The biological information providing apparatus according to claim 4, wherein
the bioimpedance signal measurement unit includes an AC signal supply unit which supplies an AC signal between a pair of electrodes in contact with the living body, and
the control unit performs control to suppress the power consumption by stopping supply of the AC signal by the AC signal supply unit.

6. The biological information providing apparatus according to claim 1, wherein
the bioimpedance signals of the first biological information output unit and the second biological information output unit or the biopotential signals of the first biological information output unit and the second biological information output unit are input to the determination unit, and
the determination unit outputs a determination signal based on the bioimpedance signals or the biopotential signals having been input.

7. The biological information providing apparatus according to claim 1, wherein
the determination unit
outputs the determination signal indicating the first determination result according to the predetermined condition in a case where a similarity degree between the bioimpedance signal of the first biological information output unit and the bioimpedance signal of the second biological information output unit is equal to or greater than a predetermined similarity degree, and
outputs the determination signal indicating the second determination result according to the predetermined condition in a case where a similarity degree between the bioimpedance signal of the first biological information output unit and the bioimpedance signal of the second biological information output unit is less than the predetermined similarity degree.

8. The biological information providing apparatus according to claim 1, wherein
the determination unit
outputs the determination signal indicating the first determination result according to the predetermined condition in a case where a similarity degree between the biopotential signal of the first biological information output unit and the biopotential signal of the second biological information output unit is equal to or greater than a predetermined similarity degree, and
outputs the determination signal indicating the second determination result according to the predetermined condition in a case where a similarity degree between the biopotential signal of the first biological information output unit and the biopotential signal of the second biological information output unit is less than the predetermined similarity degree.

9. The biological information providing apparatus according to claim 1, wherein
the determination unit
acquires index information indicating a magnitude of vibration of the living body,
outputs the determination signal indicating the first determination result according to the predetermined condition in a case where the index information indicates that a magnitude of vibration of the living body is equal to or greater than a predetermined magnitude, and
outputs the determination signal indicating the second determination result according to the predetermined condition in a case where the index information indicates that a magnitude of vibration of the living body is less than the predetermined magnitude.

10. The biological information providing apparatus according to claim 9, wherein
the determination unit
acquires, as the index information, at least one of position information of the living body, vibration information including the magnitude of vibration of the living body, or acceleration information of the living body.

11. The biological information providing apparatus according to claim 1, wherein
the biological information providing apparatus is operable in a first mode in which the second biological information output unit outputs the biopotential signal with first reliability and a second mode in which the second biological information output unit outputs the biopotential signal with second reliability higher than the first reliability, and
the determination unit
outputs the determination signal indicating the first determination result according to the predetermined condition in a case where the biological information providing apparatus operates in the first mode, and
outputs the determination signal indicating the second determination result according to the predetermined condition in a case where the biological information providing apparatus operates in the second mode.

12. The biological information providing apparatus according to claim 1, wherein the second biological information output unit includes an amplification unit which amplifies or attenuates the noise signal, which serves as an input to the biopotential signal output unit of the second biological information output unit, with an amplification factor based on a level ratio between the bioimpedance signal measured by the bioimpedance signal measurement unit of the first biological information output unit and the bioimpedance signal measured by the bioimpedance signal measurement unit of the second biological information output unit, and then outputs a resultant signal to the biopotential signal output unit of the second biological information output unit.

13. The biological information providing apparatus according to claim 1, wherein
the biological information providing apparatus includes a plurality of second biological information output units including the second biological information output unit, and
the determination unit outputs the determination signal to each of the plurality of second biological information output units according to the predetermined condition.

14. The biological information providing apparatus according to claim 13, further comprising a band which holds the pair of electrodes of each of the first biological information output unit and the plurality of second biological information output units and is worn on the living body.

15. The biological information providing apparatus according to claim 1, wherein
the biological information providing apparatus includes a plurality of first biological information output units including the first biological information output unit and a plurality of second biological information output units including the second biological information output unit,
at least one second biological information output unit of the plurality of second biological information output units is associated with each of the plurality of first biological information output units, and
in the case where the determination signal indicates the first determination result, the control unit of each of the plurality of second biological information output units performs control such that the noise signal generated by the noise signal generation unit of the first biological information output unit associated with the corresponding second biological information output unit is input to the biopotential signal output unit of the corresponding second biological

16. The biological information providing apparatus according to claim 15, further comprising a band which holds the pair of electrodes of each of the plurality of first biological information output units and the plurality of second biological information output units and is worn on the living body.

17. The biological information providing apparatus according to claim 1, wherein
the first biological information output unit further includes
a first phase delay unit which synchronizes the biopotential signal output by the biopotential signal output unit of the second biological information output unit with the biopotential signal output by the biopotential signal output unit of the corresponding first biological information output unit, and
the second biological information output unit further includes
a second phase delay unit which synchronizes the biological signal with the noise signal generated by the noise signal generation unit of the first biological information output unit.

18. A biological information providing apparatus comprising:
a plurality of biological information output units each including
a biological signal measurement unit which measures a biological signal through a pair of electrodes in contact with a same living body,
a bioimpedance signal measurement unit which measures a bioimpedance generated between the pair of electrodes to output a bioimpedance signal corresponding to the bioimpedance,
a noise signal generation unit which generates, from the bioimpedance signal, a noise signal indicating a noise component included in the biological signal, and
a biopotential signal output unit which removes a component of the noise signal from the biological signal to output a resultant signal as a biopotential signal; and
an identification information output unit which groups the plurality of biological information output units based on a similarity degree of the bioimpedance signals of the plurality of biological information output units, and outputs identification information for identifying a specific biological information output unit for each group, wherein
the plurality of biological information output units each further include
a control unit which performs control such that in a case where the corresponding biological information output unit is not a specific biological information output unit indicated by the identification information, a noise signal generated by the noise signal generation unit of the specific biological information output unit indicated by the identification information is input to its own biopotential signal output unit, and
in a case where the corresponding biological information output unit is a specific biological information output unit indicated by the identification information, a noise signal generated by its own noise signal generation unit is input to its own biopotential signal output unit.

19. A biological information providing apparatus comprising:
a plurality of biological information output units each including
a biological signal measurement unit which measures a biological signal through a pair of electrodes in contact with a same living body,
a bioimpedance signal measurement unit which measures a bioimpedance generated between the pair of electrodes to output a bioimpedance signal corresponding to the bioimpedance,
a noise signal generation unit which generates, from the bioimpedance signal, a noise signal indicating a noise component included in the biological signal, and
a biopotential signal output unit which removes a component of the noise signal from the biological signal to output a resultant signal as a biopotential signal; and
an identification information output unit which outputs identification information for identifying a specific biological information output unit among the plurality of biological information output units based on a predetermined condition, wherein
the plurality of biological information output units each further include
a control unit which performs control such that in a case where the corresponding biological information output unit is not a specific biological information output unit indicated by the identification information, a noise signal generated by the noise signal generation unit of the specific biological information output unit indicated by the identification information is input to its own biopotential signal output unit, and
in a case where the corresponding biological information output unit is a specific biological information output unit indicated by the identification information, a noise signal generated by its own noise signal generation unit is input to its own biopotential signal output unit.

20. The biological information providing apparatus according to claim 19, wherein
the biological information providing apparatus is operable in a first mode in which the plurality of biological information output units output the biopotential signal with first reliability and a second mode in which the plurality of biological information output units output the biopotential signal with second reliability higher than the first reliability,
in a case of operation in the first mode, the identification information output unit outputs the identification information to each of the plurality of biological information output units, and
in a case of operation in the second mode, the control unit performs control such that a noise signal generated by its own noise signal generation unit is input to its own biopotential signal output unit.

21. The biological information providing apparatus according to claim 19, wherein
the biological information providing apparatus is operable in a first mode in which the plurality of biological information output units output the biopotential signal with first reliability, a second mode in which the plurality of biological information output units output the biopotential signal with second reliability higher than the first reliability, and a third mode in which the plurality of biological information output units output the biopotential signal with third reliability higher than the second reliability,
the plurality of biological information output units are grouped into a first number of first groups and is also grouped into a second number of second groups, the second number being larger than the first number,
the identification information output unit
outputs the identification information indicating a specific biological information output unit for each of the first groups to each of the plurality of biological information output units in a case where the biological information providing apparatus operates in the first mode, and
outputs the identification information indicating a specific biological information output unit for each of the second groups to each of the plurality of biological information output units in a case where the biological information providing apparatus operates in the second mode, and
in a case where the biological information providing apparatus operates in the third mode, the control unit performs control such that a noise signal generated by its own noise signal generation unit is input to its own biopotential signal output unit.

22. A biological information output circuit comprising:
a biological signal measurement unit which measures a biological signal through a pair of electrodes in contact with a same living body;
a bioimpedance signal measurement unit which measures a bioimpedance generated between the pair of electrodes to output a bioimpedance signal corresponding to the bioimpedance;
a noise signal generation unit which generates, from the bioimpedance signal, a noise signal indicating a noise component included in the biological signal;
a biopotential signal output unit which removes a component of the noise signal from the biological signal to output a resultant signal as a biopotential signal; and
a control unit which performs control such that in a case where a determination signal output by a determination unit based on a predetermined condition indicates a first determination result, an input external noise signal is input to the biopotential signal output unit, and
in a case where the determination signal indicates a second determination result, the noise signal generated by the noise signal generation unit is input to the biopotential signal output unit.
